(19) United States
(12) Patent Application Publication
(10) Pub. No.: US 2025/0375477 A1
(43) Pub. Date: Dec. 11, 2025

(54) SELECTIVE TOLERIZATION - METHODS OF SELECTIVELY GENERATING TOLEROGENIC DENDRITIC CELLS

(71) Applicants: TRANSIMMUNE AG, Düsseldorf (DE); YALE UNIVERSITY, New Haven, CT (US)

(72) Inventors: Richard EDELSON, New Haven, CT (US); Karsten HENCO, Düsseldorf (DE); Olga SOBOLEV, Düsseldorf (DE); Douglas HANLON, New Haven, CT (US); Aaron VASSALL, New Haven, CT (US); Kazuki TATSUNO, New Haven, CT (US); Patrick HAN, New Haven, CT (US)

(73) Assignees: TRANSIMMUNE AG, Düsseldorf (DE); YALE UNIVERSITY, New Haven, CT (US)

(21) Appl. No.: 18/580,097

(22) PCT Filed: Jul. 29, 2022
(86) PCT No.: PCT/EP2022/071403

§ 371 Date: Jan. 17, 2024

Related U.S. Application Data
(60) Provisional application No. 63/203,726, filed on Jul. 29, 2021.

(30) Foreign Application Priority Data
Sep. 10, 2021 (EP) 21196099.2

(51) Int. Cl. A61K 35/15; A61K 40/19; A61K 40/41; A61P 37/06; C12N 5/00; C12N 5/0784

(52) U.S. Cl. CPC A61K 35/15; A61K 40/19; A61K 40/418; A61P 37/06; C12N 5/0018; C12N 5/064; C12N 2501/48; C12N 2502/1121; C12N 2529/10

(57) ABSTRACT

The present invention relates to methods of selectively generating tolerogenic dendritic cells. The present invention further relates to patient-specific tolerogenic dendritic cells obtained by the described methods which reduce immunogenicity of a transplant when administered prior to transplantation. The present invention also relates to patient-specific tolerogenic dendritic cells for use in reducing or preventing inflammatory conditions such as graft-versus-host disease. Specifically, the methods can be used to reduce graft versus host disease. The tolerogenic dendritic cells of the present invention can also be used for the treatment of autoimmune diseases.

### FIELD OF THE INVENTION

The present invention relates to methods for selectively producing tolerogenic dendritic cells. The present invention further relates to methods of reducing immunogenicity of a transplant prior to transplantation by generating tolerogenic dendritic cells. The present invention also relates to tolerogenic dendritic cells, including tolerogenic dendritic cells obtained by the described methods. The tolerogenic dendritic cells reduce immunogenicity of a transplant when administered prior to transplantation. The present invention also relates to tolerogenic dendritic cells for use in reducing or preventing inflammatory conditions such as graft-versus-host disease and/or autoimmune diseases. Specifically, the tolerogenic dendritic cells can be used to reduce graft versus host disease.

### BACKGROUND OF THE INVENTION

Transplantation of organs, tissue or cells from one genetically distinct person (donor) to another (recipient) remains the definitive therapy for several diseases but is limited by organ and donor availability. A suitable donor is an individual with an identical or near identical profile of cell-surface antigens known as major histocompatibility antigens (MHC) or HLA antigens. A transplant from the same individual (autologous transplant or autograft) is, however, not always available and widespread application of transplants from a different individual (allogeneic transplant or allograft) is limited by differences in MHC or HLA antigens. There are many alternative forms (alleles) of each of the HLA antigens, and thus the chance of two unrelated individuals being closely HLA-matched is extremely small. The adverse reactions following transplantation of an organ or tissue from one genetically distinct individual to another can be profoundly dangerous. The primary adverse reaction is immunologic rejection of the transplanted organ or tissue. This may be caused by immune system of recipient (organ, skin etc.) attacking the transplant. In addition, the transplant may also attack the recipient (GvHD). In order to prevent or limit the rejection, patients typically receive a combination of immunosuppressive drugs. These drugs are usually globally immunosuppressive, thereby greatly increasing the susceptibility of the recipient to serious infections. These adverse effects have stimulated the search for therapies that can more selectively suppress the rejection of transplanted tissue, while leaving the remainder of the immune system intact and not injuring other important organs. One approach to reverse the rejection of transplanted organs has been the application of Extracorporeal Photopheresis (ECP), a process which involves the treatment of blood with a DNA-crosslinking agent such as 8-MOP, and UV light. One possible mechanism which would explain the positive effects of ECP in the treatment of graft versus host disease (GvHD) is that monocytes contained in a blood sample will differentiate into immuno-suppressive dendritic cells upon exposure to the combination of 8-MOP and UV light. These immuno-suppressive dendritic cells are assumed to promote immune tolerance. However, it would be of great value to improve the selective tolerization of allogeneic transplants in order to increase the pool of suitable donors, advance the therapy and/or prevent autoimmune diseases, in particular graft-versus-host disease, and to further decipher possible mechanisms behind immuno-suppressive effects of ECP and ECP-like processes.

### OBJECTIVES AND SUMMARY OF THE INVENTION

One objective of the present invention is to provide methods for selectively producing tolerogenic dendritic cells. Another objective is to provide methods for selectively producing antigen-specific tolerogenic dendritic cells. Another objective is to provide methods for selectively producing tolerogenic dendritic cells which reduce immunogenicity of a transplant prior to transplantation.

Another objective of the present invention is to provide tolerogenic dendritic cells including ex vivo tolerogenic dendritic cells.

Another objective of the present invention is to provide tolerogenic dendritic cells obtained by the methods of the invention.

Another objective of the present invention is to provide tolerogenic dendritic cells for use in preventing or reducing GvHD.

Another objective of the present invention is to provide tolerogenic dendritic cells for use in preventing or reducing the rejection of organ transplants, such as e.g. the skin.

It is still another objective to provide methods of treating GvHD.

Another objective of the present invention is to provide tolerogenic dendritic cells for use in treating an autoimmune disease.

It is yet another objective to provide methods of treating an autoimmune disease.

These and other objectives as they will become apparent from the ensuing description hereinafter are solved by the subject matter of the independent claims. Some of the preferred embodiments of the present invention form the subject matter of the dependent claims. Yet other embodiments of the present invention may be taken from the ensuing description.

The present invention as illustratively described in the following may suitably be practiced in the absence of any element or elements, limitation or limitations, not specifically disclosed herein.

The present invention is described with respect to particular embodiments below and with reference to certain figures but the invention is not limited thereto but only by the claims.

The present invention is based to some extent on data and clinical trials presented hereinafter, which lead to the insight that an apoptotic dendritic cell can tolerize the immune system of a future transplant recipient against an allogeneic transplant if the apoptotic dendritic cell is taken up by a healthy dendritic cell. Surprisingly, the inventors found that the apoptotic dendritic cell may be derived from a donor or a future recipient. Also, in case the apoptotic dendritic cell is derived from a donor, the donor may be HLA matched or mismatched relative to the future recipient. Preferably, healthy dendritic cells are produced in vitro in the context of the present invention in an ECP derived process. The inventors observed that dendritic cells can be efficiently generated when leukapheresed blood samples, containing monocytes and platelets, are passed through a plate. However, the blood sample must at least contain monocytes. It has been found that monocytes mature into healthy dendritic cells upon the application of shear stress. If platelets are present, the maturation process can be improved. Importantly, the monocytes can be matured into healthy dendritic cells using this method without the need for adding expensive cytokine cocktails. As the above described process mimics some of the aspects which are assumed to take place in vivo (see Han et al., 2020, “Platelet P-selectin initiates cross-presentation and dendritic cell differentiation in blood monocytes”, Science Advances), the dendritic cells generated by plate passing are termed “physiological dendritic cells” (phDC) in the following.

It is thus hypothesized that apoptotic dendritic cells, derived from a transplant donor or future recipient, provide an antigen source to phDC from the future recipient thereby initiating an efficient tolerogenic immune response. The present invention facilitates and improves this process by bringing apoptotic dendritic cells in direct contact with phDC. The direct incubation allows an improved tolerization of the future recipient to an allogeneic transplant upon administration of the selectively produced tolerogenic dendritic cells and thus, to reduce or eliminate inflammatory conditions such as GvHD. The phDC of the present invention are more efficient than dendritic cells produced by other methods such as exposure to cytokine cocktails. Moreover, the phDC can be produced in a standardized and reproducible manner leading to a better control of the process of generating potent tolerogenic dendritic cells. Further, the phDC can be used in treatment of autoimmune diseases.

The above described selective generation of tolerogenic dendritic cells can be exploited in different ways which are described in the following as the first, second and third aspect.

### First Aspect: a Method to Selectively Produce Tolerogenic Dendritic Cells, Wherein the Donor Dendritic Cells are Rendered Apoptotic

In a first aspect, the invention relates to a method comprising the following steps:
a) Providing dendritic cells from a donor;
b) Exposing the dendritic cells of step a) to apoptotic agents;
c) Providing physiologic dendritic cells from a recipient; and
d) Combining the apoptotic donor dendritic cells of step b) with physiologic recipient dendritic cells from step c).

In one embodiment, the method is performed prior to transplantation.

In one embodiment the method is for selectively reducing immunogenicity of a transplant or a part thereof prior to transplantation.

In one embodiment, the above method steps are performed simultaneously.

In one embodiment, the above method steps are performed sequentially. Thus, the phDC from the recipient are not exposed to apoptotic agents. In one embodiment, the phDC from the recipient are not exposed to apoptotic agents at any time point during the method.

In one embodiment, the dendritic cells of step a) are obtained from a donor.

In one embodiment, the dendritic cells of step c) are obtained from a recipient.

In principle, after the dendritic cells have been obtained from the donor, donor dendritic cells can be viable or not. Donor dendritic cells could e.g. be made apoptotic and stored frozen until they are combined with the physiologic recipient dendritic cells from step c).

In one embodiment, the donor is allogeneic. In one embodiment, the donor is a haplo-donor.

In one embodiment, the invention relates to a method comprising the following steps:
a) Providing dendritic cells from a donor;
b) Exposing the dendritic cells of step a) to apoptotic agents;
c) Providing physiologic dendritic cells from a recipient; and
d) Combining the apoptotic donor dendritic cells of step b) with physiologic recipient dendritic cells from step c), and
d1) Co-incubating the mixture of step d).

In one embodiment, step d1) of co-culturing the mixture is performed for at least 0.5 h, 1 h, 2 h, 3 h, 4 h, 5 h or 6 h.

In one embodiment, step d) of combining the apoptotic donor dendritic cells with the physiologic dendritic cells from the recipient takes place within the recipient.

In one embodiment, the above method steps are performed simultaneously.

In one embodiment, the above method steps are performed sequentially. Thus, the phDC from the recipient are not exposed to apoptotic agents.

In step b) of the method, the dendritic cells which are obtained from a donor in method step a) are exposed to apoptotic agents. In one embodiment, the apoptotic agents comprise psoralens and UVA, riboflavin-phosphate and UVA and/or aminolevulinic acid and light. Particularly preferred psoralens are 8-MOP and amotosalen. The most preferred psoralen is 8-MOP. In a most preferred embodiment, the apoptotic agents are the combination of 8-MOP and UVA.

Embodiments should preferably be chosen so that essentially all dendritic cells from the donor are in contact with the apoptotic agents. In case of 8-MOP/UVA, essentially all dendritic cells from the donor should be in contact with 8-MOP and exposed to UVA light. Typical doses of 8-MOP and UVA are 1 J/cm² to 3 J/cm² UVA in combination with a concentration of 8-MOP of 100 ng/ml to 300 ng/mL.

In preferred embodiments, the dose of UVA is equal to or below 3 J/cm², equal to or below 2 J/cm², or equal to or below 1 J/cm². In other preferred embodiments, the dose of 8-MOP is equal to or below 300 ng/ml, 250 ng/mL, 200 ng/ml or 100 ng/mL. In a particularly preferred embodiment, the dose of 8-MOP is 200 ng/ml and the dose of UVA is 1 J/cm².

In the context of the invention dendritic cells may in particular be obtained by plate-passage of monocytes using an extracorporeal photopheresis (ECP) derived process.

Methods and devices for extracorporeal activation of monocytes and generation of dendritic cells therefrom are described in WO2014/106629 A1, WO2014/106631 A1, WO2016/001405 A1, and WO2017/005700 A1, each of which is incorporated herein by reference in its entirety. ECP describes a process in which monocytes derived from a blood sample or a fraction thereof are exposed to mechanical stress (e.g., shear forces) and plasma components (e.g., platelets) or derivatives or mimics thereof, thereby activating the monocytes to differentiate into healthy, physiologic dendritic cells which are also termed phDC herein. ECP and ECP derived processes, including the differentiation of monocytes into phDC, may be performed in a large-scale ECP device, e.g., a clinical ECP device (e.g., a THERAKOS® CELLEX® device), or in a miniaturized ECP device, e.g., a Transimmunization plate as described in WO2017/005700 A1; or a bag such as a plastic bag (e.g. a plastic bag for blood, blood components, cell therapies etc.).

The inventors found that phDC obtained by the method described above, are advantageous as compared to DC obtained by other methods such as cytokines or direct isolation from the recipient, as phDC are generated physiologically (without the need for chemicals such as cytokines) with greater reproducibility and controllability under precise in vitro laboratory conditions.

Thus, in an especially preferred embodiment, phDC of the recipient are obtained by subjecting monocytes contained in a blood sample to a shear force by passing the blood sample or fraction thereof through a flow chamber of a device. Preferably, platelets are present in the flow chamber which can be either derived from the recipient's blood sample or a fraction thereof or provided separately. Additionally or alternatively, plasma components can be present in the flow chamber which can be either derived from the recipient's blood sample or a fraction thereof or provided separately. However, the generation of phDC also works in the absence of platelets and/or plasma components.

A monocyte of a recipient may be obtained by any suitable means, e.g., from a blood sample or a fraction thereof. The fraction of the blood sample may be, e.g., a buffy coat including white blood cells and platelets. Alternatively, the fraction of the blood sample may be an isolated peripheral blood mononuclear cell (PBMC). PBMCs may be isolated from a blood sample using, e.g., centrifugation over a Ficoll-Hypaque gradient (Isolymph, CTL Scientific). In another example, the fraction of the blood sample may be a purified or enriched monocyte preparation. Monocytes may be enriched from PBMCs using, e.g., one, two, or all three of plastic adherence; CD14 magnetic bead positive selection (e.g., from Miltenyi Biotec); and a Monocyte Isolation Kit II (Miltenyi Biotec).

Any suitable volume of blood can be used. The blood sample (e.g., the blood sample from which the fraction is derived) may be between about 1 μL and about 500 mL, e.g., between about 1 μL and about 10 mL, between about 1 μL and about 5 mL, between about 1 μL and about 1 mL, between about 1 μL and about 750 μL, between about 1 μL and about 500 μL, between about 1 μL and about 250 μL, between about 10 mL and about 450 mL, about 20 mL and about 400 mL, about 30 mL and about 350 mL, about 40 mL and about 300 mL, about 50 mL and about 200 mL, or about 50 mL and about 100 mL. In some embodiments, the blood sample or the fraction thereof or the additional blood sample or the fraction thereof is less than or equal to about 100 mL (e.g., about 50 mL to about 100 mL).

In some embodiments, the ECP device is a miniaturized ECP device, e.g. a transimmunization (TI) plate. In some embodiments, the ECP device is a plastic bag. The skilled person is well aware of methods to distinguish dendritic cells including phDC from monocytes, such as e.g. by assessing gene expression.

Without being bound to a scientific theory, the inventors assume at present that the remarkable effects of the present invention are due to damaged dying DCs, in particular damaged by apoptotic agents such as the combination of a psoralen and UVA (PUVA), particularly 8-MOP and UVA, which provide antigens to the physiologic DCs of the recipient and a tolerance signal to the immune system of the recipient. If phDCs have received such tolerogenic signals from PUVA-treated apoptotic DCs which are allogeneic, the phDCs (in addition to the received tolerogenic signal) may display antigens from the allogeneic PUVA-treated apoptotic DCs on their surface and are thereby capable of triggering an antigen-specific tolerogenic response. Analogous, the source of the antigen may also be derived from immune cells, e.g. monocytes or lymphocytes. Thus, immune cells, e.g. monocytes or lymphocytes, may be rendered apoptotic and combined with phDCs in order to generate an antigen-specific tolerogenic response. However, damaged DCs or related precursor cells such as monocytes are preferred.

In one embodiment, the dendritic cells of step a) are derived from an extracorporeal blood sample of the donor. In another embodiment, the dendritic cells of step a) have been obtained by plate passage of PBMC from the donor. Thus, the dendritic cells of the donor may also be phDC. All embodiments relating to the provision of an extracorporeal blood sample and PBMCs as described above with respect to the recipient also apply to the donor.

In one embodiment, the recipient and donor are mammalian. Mammals include for example, but are not limited to, humans, non-human primates, pigs, dogs, cats, horses and rodents. In a preferred embodiment, the recipient and donor are human.

In one embodiment, the transplant is a kidney transplant, pancreas transplant, liver transplant, heart transplant, lung transplant, bowel transplant, skin transplant, bone marrow transplant or a stem cell transplant.

In one embodiment, the stem cell transplant is a hematopoietic stem cell transplant.

All of the above embodiments may be carried out in vitro.

The methods of the present invention can be applied in conjunction with other therapies for the treatment of immune defects associated with hematopoietic stem cell transplantation.

For the following embodiment of the first aspect, all of the above embodiments relating to the first aspect apply mutatis mutandis:

In one embodiment, the invention relates to a method comprising the following steps:
a) Exposing dendritic cells obtained from a donor to apoptotic agents;
b) Combining the apoptotic donor dendritic cells of step a) with physiologic dendritic cells which have been obtained from a recipient.

In the above embodiment, a co-incubation may be carried out which corresponds to step d1) as described for the embodiments further above.

In one embodiment, the above method steps are performed simultaneously.

In one embodiment, the above method steps are performed sequentially. Thus, the phDC obtained from the recipient are not exposed to apoptotic agents. In one embodiment, the phDC from the recipient are not exposed to apoptotic agents at any time point during the method.

In one embodiment, the invention relates to a method comprising the following steps:
a) Providing immune cells, preferably lymphocytes, from a donor;
b) Exposing the immune cells, preferably lymphocytes, of step a) to apoptotic agents;
c) Providing physiologic dendritic cells from a recipient; and
d) Combining the apoptotic immune cells, preferably apoptotic lymphocytes, of step b) with physiologic recipient dendritic cells from step c).

All of the embodiments of the first aspect apply mutatis mutandis to the above embodiment (i.e. with the dendritic cells obtained from a donor being replaced by immune cells, preferably lymphocytes, obtained from a donor).

The method can also be practiced based on dendritic cell related cells such as monocytes being rendered apoptotic. Thus, in another embodiment, the invention relates to a method comprising the following steps:
a) Providing monocytes from a donor;
b) Exposing the monocytes of step a) to apoptotic agents;
c) Providing physiologic dendritic cells from a recipient; and
d) Combining the apoptotic monocytes of step b) with physiologic recipient dendritic cells from step c).

All of the embodiments of the first aspect apply mutatis mutandis to the above embodiment (i.e. with the dendritic cells obtained from a donor being replaced by monocytes obtained from a donor).

### Second Aspect: a Method to Selectively Produce Tolerogenic Dendritic Cells, Wherein the Complimentary Haplo-Donor Dendritic Cells are Rendered Apoptotic

In a second aspect, the invention relates to a method comprising the following steps:
a) Providing dendritic cells from a recipient's complimentary haplo-donor;
b) Exposing the dendritic cells of step a) to apoptotic agents;
c) Providing physiologic dendritic cells from the recipient's haplo-donor; and
d) Combining the apoptotic complimentary haplo-donor dendritic cells of step b) with the physiologic haplo-donor dendritic cells of step c).

In one embodiment, the method is performed prior to transplantation.

In one embodiment the method is for selectively reducing immunogenicity in a transplant or a part thereof prior to transplantation.

In one embodiment, the above method steps are performed simultaneously.

In one embodiment, the above method steps are performed sequentially. Thus, the phDC from the recipient's haplo-donor are not exposed to apoptotic agents. In one embodiment, the phDC from the recipient's haplo-donor are not exposed to apoptotic agents at any time point of the method.

In one embodiment, the dendritic cells of step a) are obtained from a recipient's complimentary haplo-donor.

In one embodiment, the dendritic cells of step c) are obtained from a recipient's haplo-donor.

In principle, after the dendritic cells have been obtained from the complimentary haplo-donor, the dendritic cells can be viable or not. Complimentary haplo-donor dendritic cells could e.g. be made apoptotic and stored frozen until they are combined with the physiologic haplo-donor dendritic cells from step c).

In one embodiment, the invention relates to a method comprising the following steps:
a) Providing dendritic cells from a recipient's complimentary haplo-donor;
b) Exposing the dendritic cells of step a) to apoptotic agents;
c) Providing physiologic dendritic cells from the recipient's haplo-donor; and
d) Combining the apoptotic complimentary haplo-donor dendritic cells of step b) with the physiologic haplo-donor dendritic cells of step c); and
d1) Co-incubating the mixture of step d).

In one embodiment, step d1) of co-culturing the mixture is performed for at least 0.5 h, 1 h, 2 h, 3 h, 4 h, 5 h or 6 h.

In one embodiment, step d) of combining the apoptotic complimentary haplo-donor dendritic cells of step b) with the physiologic dendritic cells from the haplo-donor takes place within the haplo-donor.

In one embodiment, the above method steps are performed simultaneously.

In one embodiment, the above method steps are performed sequentially. Thus, the phDC from the recipient's haplo-donor are not exposed to apoptotic agents. In one embodiment, the phDC from the recipient's haplo-donor are not exposed to apoptotic agents at any time point of the method.

In step b) of the method, the dendritic cells which are obtained from a recipient's complimentary haplo-donor in method step a) are exposed to apoptotic agents. In one embodiment, the apoptotic agents comprise psoralens and UVA, riboflavin-phosphate and UVA and/or aminolevulinic acid and light. Particularly preferred psoralens are 8-MOP and amotosalen. The most preferred psoralen is 8-MOP. In a most preferred embodiment, the apoptotic agents are the combination of 8-MOP and UVA.

Embodiments should preferably be chosen so that essentially all dendritic cells from the recipient's complimentary haplo-donor are in contact with the apoptotic agents. In case of 8-MOP/UVA, essentially all dendritic cells from the recipient's complimentary haplo-donor should be in contact with 8-MOP and exposed to UVA light. Typical doses of 8-MOP and UVA are 1 J/cm² to 3 J/cm² UVA in combination with a concentration of 8-MOP of 100 ng/mL to 300 ng/mL.

In preferred embodiments, the dose of UVA is equal to or below 3 J/cm², equal to or below 2 J/cm², or equal to or below 1 J/cm². In other preferred embodiments, the dose of 8-MOP is equal to or below 300 ng/ml, 250 ng/ml, 200 ng/ml or 100 ng/mL. In a preferred embodiment, the dose of 8-MOP is 200 ng/ml and the dose of UVA is 1 J/cm².

As described with respect to the first aspect, dendritic cells in the context of the present invention may in particular be obtained by plate-passage of monocytes using an extracorporeal photopheresis (ECP) derived process, activating monocytes to differentiate into healthy phDC. All embodiments relating to the generation of phDC as described for the first aspect also apply to the generation of phDC in the second aspect.

Thus, in an especially preferred embodiment, phDC of the haplo-donor are obtained by subjecting monocytes contained in a blood sample to a shear force by passing the blood sample or fraction thereof through a flow chamber of a device. Preferably, platelets are present in the flow chamber which can be either derived from the haplo-donor's blood sample or a fraction thereof or provided separately. Additionally or alternatively, plasma components can be present in the flow chamber which can be either derived from the haplo-donor's blood sample or a fraction thereof or provided separately. However, the generation of phDC also works in the absence of platelets and/or plasma components.

A monocyte of a haplo-donor may be obtained by any suitable means, e.g., from a blood sample or a fraction thereof. The fraction of the blood sample may be, e.g., a buffy coat including white blood cells and platelets. Alternatively, the fraction of the blood sample may be an isolated peripheral blood mononuclear cell (PBMC). PBMCs may be isolated from a blood sample using, e.g., centrifugation over a Ficoll-Hypaque gradient (Isolymph, CTL Scientific). In another example, the fraction of the blood sample may be a purified or enriched monocyte preparation. Monocytes may be enriched from PBMCs using, e.g., one, two, or all three of plastic adherence; CD14 magnetic bead positive selection (e.g., from Miltenyi Biotec); and a Monocyte Isolation Kit II (Miltenyi Biotec).

Any suitable volume of blood can be used. The blood sample (e.g., the blood sample from which the fraction is derived) may be between about 1 μL and about 500 mL, e.g., between about 1 μL and about 10 mL, between about 1 μL and about 5 mL, between about 1 μL and about 1 mL, between about 1 μL and about 750 μL, between about 1 μL and about 500 μL, between about 1 μL and about 250 μL, between about 10 mL and about 450 mL, about 20 mL and about 400 mL, about 30 mL and about 350 mL, about 40 mL and about 300 mL, about 50 mL and about 200 mL, or about 50 mL and about 100 mL. In some embodiments, the blood sample or the fraction thereof or the additional blood sample or the fraction thereof is less than or equal to about 100 mL (e.g., about 50 mL to about 100 mL).

In some embodiments, the ECP device is a miniaturized ECP device, e.g. a transimmunization (TI) plate. In some embodiments, the ECP device is a plastic bag. The skilled person is well aware of methods to distinguish dendritic cells including phDC from monocytes, such as e.g. by assessing gene expression.

Without being bound to a scientific theory, the inventors assume at present that the remarkable effects of the present invention are due to damaged dying DCs, in particular damaged by apoptotic agents such as the combination of a psoralen and UVA (PUVA), in particular 8-MOP and UVA, which provide antigens to the physiologic DCs of the haplo-donor and a tolerance signal to the immune system of the haplo-donor. If phDCs have received such tolerogenic signals from PUVA-treated apoptotic DCs from the complimentary haplo-donor, the phDCs (in addition to the received tolerogenic signal) may display antigens from the PUVA-treated apoptotic DCs on their surface and thereby trigger an antigen-specific tolerogenic response in the haplo-donor. Inflammatory conditions such as GvHD will be reduced or eliminated in a recipient, receiving a transplant from the haplo-donor treated as described in the second aspect. Analogous, the source of the antigen may also be derived from immune cells, e.g. lymphocytes or monocytes. Thus, immune cells, e.g. lymphocytes or monocytes, may be rendered apoptotic and combined with haplo-donor phDCs in order to generate an antigen-specific tolerogenic response. However, damaged DCs or related precursor cells such as monocytes are preferred.

In one embodiment, the dendritic cells of step a) are derived from an extracorporeal blood sample of the recipient's complimentary haplo-donor. In another embodiment, the dendritic cells of step a) have been obtained by plate passage of PBMC from the recipient's complimentary haplo-donor. Thus, the dendritic cells of the recipient's complimentary haplo-donor may also be phDC.

In one embodiment, the complimentary haplo-donor and haplo-donor are mammalian. Mammals include for example, but are not limited to, humans, non-human primates, pigs, dogs, cats, horses and rodents. In a preferred embodiment, the complimentary haplo-donor and haplo-donor are human.

In one embodiment, the transplant is a kidney transplant, pancreas transplant, liver transplant, heart transplant, lung transplant, bowel transplant, bone marrow transplant or a stem cell transplant.

In one embodiment, the stem cell transplant is a hematopoietic stem cell transplant.

For the following embodiment of the second aspect, all of the above embodiments relating to the first and second aspect apply mutatis mutandis:

In one embodiment, the invention relates to a method comprising the following steps:
a) Exposing dendritic cells obtained from a recipient's complimentary haplo-donor to apoptotic agents; and
b) Combining the apoptotic recipient's complimentary haplo-donor dendritic cells of step a) with physiologic dendritic cells which have been obtained from the recipient's haplo-donor.

It needs to be understood that all method steps may be performed in vitro.

In one embodiment, the above method steps are performed simultaneously.

In one embodiment, the above method steps are performed sequentially. Thus, the phDC obtained from the recipient's haplo-donor are not exposed to apoptotic agents.

In one embodiment, the phDC from the recipient's haplo-donor are not exposed to apoptotic agents at any time point of the method.

In one embodiment, the invention relates to a method comprising the following steps:
a) Providing immune cells, preferably lymphocytes, from a recipient's complimentary haplo-donor;
b) Exposing the immune cells, preferably lymphocytes, of step a) to apoptotic agents;
c) Providing physiologic dendritic cells from the recipient's haplo-donor; and
d) Combining the apoptotic complimentary haplo-donor immune cells, preferably apoptotic complimentary haplo-donor lymphocytes, of step
b) with the physiologic haplo-donor dendritic cells of step c).

All of the embodiments of the second aspect apply mutatis mutandis to the above embodiment (i.e. with the dendritic cells obtained from a recipient's complimentary haplo-donor being replaced by immune cells, preferably lymphocytes, obtained from a recipient's complimentary haplo-donor).

The method can also be practiced based on dendritic cell related cells such as monocytes being rendered apoptotic. Thus, in another embodiment, the invention relates to a method comprising the following steps:
a) Providing monocytes from a recipient's complimentary haplo-donor;
b) Exposing monocytes of step a) to apoptotic agents;
c) Providing physiologic dendritic cells from the recipient's haplo-donor; and
d) Combining the apoptotic complimentary haplo-donor monocytes of step b) with the physiologic haplo-donor dendritic cells of step c).

All of the embodiments of the first aspect apply mutatis mutandis to the above embodiment (i.e. with the dendritic cells obtained from a recipient's complimentary haplo-donor being replaced by monocytes obtained from a recipient's complimentary haplo-donor).

### Third Aspect: a Method to Selectively Produce Tolerogenic Dendritic Cells, Wherein the Recipient Dendritic Cells are Rendered Apoptotic

In a third aspect, the invention relates to a method comprising the following steps:
a) Providing dendritic cells from a recipient;
b) Exposing the dendritic cells of step a) to an apoptotic agent;
c) Providing physiologic dendritic cells from the recipient; and
d) Combining the apoptotic dendritic cells of step b) with the physiologic dendritic cells of step c).

In one embodiment, the method is performed prior to transplantation.

In one embodiment the method is for selectively reducing immunogenicity in a transplant or a part thereof prior to transplantation.

In one embodiment, the above method steps are performed simultaneously.

In one embodiment, the above method steps are performed sequentially. Thus, the phDC from the recipient (step c) are not exposed to apoptotic agents. In one embodiment, the phDC from the recipient of step c) are not exposed to apoptotic agents at any time point of the method.

In one embodiment, the dendritic cells of step a) are obtained from a recipient.

In one embodiment, the dendritic cells of step c) are obtained from the recipient.

In principle, after the dendritic cells have been obtained from the recipient (step a), the dendritic cells can be viable or not. The dendritic cells could e.g. be made apoptotic and stored frozen until they are combined with the physiologic recipient dendritic cells from step c).

In one embodiment, the invention relates to a method comprising the following steps:
a) Providing dendritic cells from a recipient;
b) Exposing the dendritic cells of step a) to an apoptotic agent;
c) Providing physiologic dendritic cells from the recipient; and
d) Combining the apoptotic dendritic cells of step b) with the physiologic dendritic cells of step c); and
d1) Co-incubating the mixture of step d).

In one embodiment, step d1) of co-culturing the mixture is performed for at least 0.5 h, 1 h, 2 h, 3 h, 4 h, 5 h or 6 h.

In one embodiment, step d) of combining the apoptotic recipient dendritic cells with the physiologic dendritic cells from the recipient takes place within the recipient.

In one embodiment, the above method steps are performed simultaneously.

In one embodiment, the above method steps are performed sequentially. Thus, the phDC from the recipient (step c) are not exposed to apoptotic agents. In one embodiment, the phDC from the recipient of step c) are not exposed to apoptotic agents at any time point of the method.

In step b) of the method, the dendritic cells which are obtained from a recipient in method step a) are exposed to apoptotic agents. In one embodiment, the apoptotic agents comprise psoralens and UVA, riboflavin-phosphate and UVA and/or aminolevulinic acid and light. Particularly preferred psoralens are 8-MOP and amotosalen. The most preferred psoralen is 8-MOP. In a most preferred embodiment, the apoptotic agents are the combination of 8-MOP and UVA.

Embodiments should preferably be chosen so that essentially all dendritic cells from the recipient are in contact with the apoptotic agents. In case of 8-MOP/UVA, essentially all dendritic cells from the recipient should be in contact with 8-MOP and exposed to UVA light. Typical doses of 8-MOP and UVA are 1 J/cm² to 3 J/cm² UVA in combination with a concentration of 8-MOP of 100 ng/ml to 300 ng/mL.

In preferred embodiments, the dose of UVA is equal to or below 3 J/cm², equal to or below 2 J/cm², or equal to or below 1 J/cm². In other preferred embodiments, the dose of 8-MOP is equal to or below 300 ng/mL, 250 ng/mL, 200 ng/ml or 100 ng/mL. In a preferred embodiment, the dose of 8-MOP is 200 ng/ml and the dose of UVA is 1 J/cm².

As described with respect to the first and second aspect, dendritic cells in the context of the present invention may in particular be obtained by plate-passage of monocytes using an extracorporeal photopheresis (ECP) derived process, activating monocytes to differentiate into healthy phDC. All embodiments relating to the generation of phDC as described for the first aspect also apply to the generation of phDC for the third aspect.

Thus, in an especially preferred embodiment, phDC of the recipient are obtained by subjecting monocytes contained in a blood sample to a shear force by passing the blood sample or fraction thereof through a flow chamber of a device. Preferably, platelets are present in the flow chamber which can be either derived from the recipient's blood sample or a fraction thereof or provided separately. Additionally or alternatively, plasma components can be present in the flow chamber which can be either derived from the recipient's blood sample or a fraction thereof or provided separately. However, the generation of phDC also works in the absence of platelets and/or plasma components.

A monocyte of a recipient may be obtained by any suitable means, e.g., from a blood sample or a fraction thereof. The fraction of the blood sample may be, e.g., a buffy coat including white blood cells and platelets. Alternatively, the fraction of the blood sample may be an isolated peripheral blood mononuclear cell (PBMC). PBMCs may be isolated from a blood sample using, e.g., centrifugation over a Ficoll-Hypaque gradient (Isolymph, CTL Scientific). In another example, the fraction of the blood sample may be a purified or enriched monocyte preparation. Monocytes may be enriched from PBMCs using, e.g., one, two, or all three of plastic adherence; CD14 magnetic bead positive selection (e.g., from Miltenyi Biotec); and a Monocyte Isolation Kit II (Miltenyi Biotec).

Any suitable volume of blood can be used. The blood sample (e.g., the blood sample from which the fraction is derived) may be between about 1 μL and about 500 mL, e.g., between about 1 μL and about 10 mL, between about 1 μL and about 5 mL, between about 1 μL and about 1 mL, between about 1 μL and about 750 μL, between about 1 μL and about 500 μL, between about 1 μL and about 250 μL, between about 10 mL and about 450 mL, about 20 mL and about 400 mL, about 30 mL and about 350 mL, about 40 mL and about 300 mL, about 50 mL and about 200 mL, or about 50 mL and about 100 mL. In some embodiments, the blood sample or the fraction thereof or the additional blood sample or the fraction thereof is less than or equal to about 100 mL (e.g., about 50 mL to about 100 mL).

In some embodiments, the ECP device is a miniaturized ECP device, e.g. a transimmunization (TI) plate. In some embodiments, the ECP device is a plastic bag. The skilled person is well aware of methods to distinguish dendritic cells including phDC from monocytes, such as e.g. by assessing gene expression.

Without being bound to a scientific theory, the inventors assume at present that the remarkable effects of the present invention are due to damaged dying DCs, in particular damaged by the combination of a psoralen and UVA (PUVA), which provide antigens to the physiologic DCs of the recipient and a tolerance signal to the immune system of the recipient. If phDCs have received such tolerogenic signals from PUVA-treated apoptotic autologous DCs, the phDCs (in addition to the received tolerogenic signal) may display antigens from the autologous PUVA-treated apoptotic DCs on their surface and thereby trigger an antigen-specific tolerogenic response. Analogous, the source of the antigen may also be derived from immune cells, e.g. monocytes or lymphocytes. Thus, immune cells, e.g. monocytes or lymphocytes, may be rendered apoptotic and combined with phDCs in order to generate an antigen-specific tolerogenic response. However, damaged DCs or related precursor cells such as monocytes are preferred.

In one embodiment, the dendritic cells of step a) are derived from an extracorporeal blood sample of the recipient. In another embodiment, the dendritic cells of step a) have been obtained by plate passage of PBMC from the recipient. In one embodiment, the recipient is mammalian. Mammals include for example, but are not limited to, humans, non-human primates, pigs, dogs, cats, horses and rodents. In a preferred embodiment, the recipient is human.

In one embodiment, the transplant is a kidney transplant, pancreas transplant, liver transplant, heart transplant, lung transplant, bowel transplant, bone marrow transplant or a stem cell transplant. In one embodiment, the stem cell transplant is a hematopoietic stem cell transplant.

All of the above embodiments may be carried out in vitro.

The methods of the present invention can be applied in conjunction with other therapies for the treatment of immune defects associated with hematopoietic stem cell transplantation.

For the following embodiment of the third aspect, all of the above embodiments relating to the third aspect apply mutatis mutandis:

In one embodiment, the invention relates to a method comprising the following steps:
a) Exposing dendritic cells obtained from a recipient to apoptotic agents;
b) Combining the apoptotic recipient dendritic cells of step a) with physiologic dendritic cells which have been obtained from the recipient.

In the above embodiment, a co-incubation may be carried out which corresponds to step d1) as described for the embodiments further above.

In one embodiment, the above method steps are performed simultaneously.

In one embodiment, the above method steps are performed sequentially. Thus, the phDC from the recipient (step b) are not exposed to apoptotic agents. In one embodiment, the phDC which have been obtained from the recipient of step b) are not exposed to apoptotic agents at any time point of the method.

In one embodiment, the invention relates to a method comprising the following steps:
a) Providing immune cells, preferably lymphocytes, from a recipient;
b) Exposing the immune cells, preferably lymphocytes, of step a) to an apoptotic agent;
c) Providing physiologic dendritic cells from the recipient; and
d) Combining the apoptotic immune cells, preferably apoptotic lymphocytes, of step b) with the physiologic dendritic cells of step c).

All of the embodiments of the third aspect apply mutatis mutandis to the above embodiment (i.e. with the dendritic cells obtained from a recipient being replaced by immune cells, preferably lymphocytes, obtained from a recipients).

The method can also be practiced based on dendritic cell related cells such as monocytes being rendered apoptotic. Thus, in another embodiment, the invention relates to a method comprising the following steps:
a) Providing monocytes from a recipient;
b) Exposing the monocytes of step a) to an apoptotic agent;
c) Providing physiologic dendritic cells from the recipient; and
d) Combining the apoptotic monocytes of step b) with the physiologic dendritic cells of step c).

All of the embodiments of the first aspect apply mutatis mutandis to the above embodiment (i.e. with the dendritic cells obtained from a recipient being replaced by monocytes obtained from a recipient).

### Fourth Aspect: Tolerogenic Dendritic Cells Obtained by a Method According to the First Aspect

In a fourth aspect, the present invention relates to tolerogenic recipient dendritic cells obtained by a method according to the first aspect (including all embodiments as described above).

In one embodiment, the tolerogenic recipient dendritic cells obtained by a method according to the first aspect reduce immunogenicity of a future transplant from the donor.

### Fifth Aspect: Tolerogenic Dendritic Cells Obtained by a Method According to the Second Aspect

In a fifth aspect, the present invention relates to tolerogenic haplo-donor dendritic cells obtained by a method according to the second aspect (including all embodiments as described above).

In one embodiment, the tolerogenic haplo-donor dendritic cells obtained by a method according to the second aspect reduce immunogenicity of a future transplant from the haplo-donor.

### Sixth Aspect: Tolerogenic Dendritic Cells Obtained by a Method According to the Third Aspect

In a sixth aspect, the present invention relates to tolerogenic recipient dendritic cells obtained by a method according to the third aspect (including all embodiments as described above).

In one embodiment, the tolerogenic recipient dendritic cells obtained by a method according to the third aspect reduce immunogenicity of a future transplant.

### Seventh Aspect: Tolerogenic Dendritic Cells According to the Fourth Aspect for Use in a Method of Preventing or Reducing Graft-Versus-Host Disease

In a seventh aspect, the present invention relates to tolerogenic dendritic cells according to the fourth aspect (including all embodiments of the first and fourth aspect as described above) for use in a method of preventing or reducing graft-versus-host disease.

In one embodiment, tolerogenic dendritic cells, which have been obtained by the methods of the first aspect, are for use in the treatment of an allogeneic or haploidentical recipient in need of a transplant. Following such a treatment, which takes place prior to transplantation, the risk of developing GvHD, and the severity of GvHD if it develops, is significantly reduced compared to not administering the tolerogenic dendritic cells prior to transplantation.

### Eighth Aspect: Tolerogenic Dendritic Cells According to the Fifth Aspect for Use in a Method of Preventing or Reducing Graft-Versus-Host Disease

In an eighth aspect, the present invention relates to tolerogenic dendritic cells according to the fifth aspect (including all embodiments of the second and fifth aspect as described above) for use in a method of preventing or reducing graft-versus-host disease.

In one embodiment, tolerogenic dendritic cells, which have been obtained by the methods of the second aspect, are for use in the treatment of a haploidentical recipient in need of a transplant. Following such a treatment, which takes place prior to transplantation, the risk of developing GvHD, and the severity of GvHD if it develops, is significantly reduced compared to not administering the tolerogenic dendritic cells prior to transplantation.

### Ninth Aspect: Tolerogenic Dendritic Cells According to the Sixth Aspect for Use in a Method of Preventing or Reducing Graft-Versus-Host Disease

In a ninth aspect, the present invention relates to tolerogenic dendritic cells according to the sixth aspect (including all embodiments of the third and sixth aspect as described above) for use in a method of preventing or reducing graft-versus-host disease.

In one embodiment, tolerogenic dendritic cells, which have been obtained by the methods of the third aspect, are for use in the treatment of an allogeneic or haploidentical recipient in need of a transplant. Following such a treatment, which takes place prior to transplantation, the risk of developing GvHD, and the severity of GvHD if it develops, is significantly reduced compared to not administering the tolerogenic recipient dendritic cells prior to transplantation.

### Tenth Aspect: a Method to Selectively Produce Tolerogenic Dendritic Cells

In a tenth aspect, the invention relates to a method comprising the following steps:
a) Providing a first sample of dendritic cells obtained from a subject;
b) Exposing the dendritic cells of step a) to an apoptotic agent;
c) Providing a second sample of dendritic cells obtained from the subject; and
d) Combining the apoptotic dendritic cells of step b) with the dendritic cells of step c).

In one embodiment, the method relates to the selective production of tolerogenic dendritic cells.

In one embodiment, step d) of combining the apoptotic dendritic cells with the dendritic cells takes place within the subject.

In one embodiment, the above method steps are performed simultaneously.

In one embodiment, the above method steps are performed sequentially. Thus, the DC of step c) are not exposed to apoptotic agents. In one embodiment, the DC of step c) are not exposed to apoptotic agents at any time point during the method.

In one embodiment, the invention relates to a method comprising the following steps:
a) Providing a first sample of dendritic cells obtained from a subject;
b) Exposing the dendritic cells of step a) to an apoptotic agent;
c) Providing a second sample of dendritic cells obtained from the subject; and
d) Combining the apoptotic dendritic cells of step b) with the dendritic cells of step c); and
d1) Co-incubating the mixture of step d).

In one embodiment, both samples of dendritic cells (step a) and c)) are obtained from the same subject. In one embodiment, step d1) of co-culturing the mixture is performed for at least 0.5 h, 1 h, 2 h, 3 h, 4 h, 5 h or 6 h.

In one embodiment, the above method steps are performed simultaneously.

In one embodiment, the above method steps are performed sequentially. Thus, the DC of step c) are not exposed to apoptotic agents.

In step b) of the method, the dendritic cells which are obtained from a subject in method step a) are exposed to apoptotic agents. In one embodiment, the apoptotic agents comprise psoralens and UVA, riboflavin-phosphate and UVA and/or aminolevulinic acid and light. Particularly preferred psoralens are 8-MOP and amotosalen. The most preferred psoralen is 8-MOP. In a most preferred embodiment, the apoptotic agents are the combination of 8-MOP and UVA.

Embodiments should preferably be chosen so that essentially all dendritic cells from the subject are in contact with the apoptotic agents. In case of 8-MOP/UVA, essentially all dendritic cells from the subject should be in contact with 8-MOP and exposed to UVA light. Typical doses of 8-MOP and UVA are 1 J/cm² to 3 J/cm² UVA in combination with a concentration of 8-MOP of 100 ng/mL to 300 ng/mL.

In preferred embodiments, the dose of UVA is equal to or below 3 J/cm², equal to or below 2 J/cm², or equal to or below 1 J/cm². In other preferred embodiments, the dose of 8-MOP is equal to or below 300 ng/mL, 250 ng/ml, 200 ng/ml or 100 ng/mL. In a preferred embodiment, the dose of 8-MOP is 200 ng/ml and the dose of UVA is 1 J/cm².

As described with respect to the first and second aspect, dendritic cells in the context of the present invention, i.e. also with respect to the tenth aspect, may in particular be obtained by plate-passage of monocytes using an extracorporeal photopheresis (ECP) derived process, activating monocytes to differentiate into healthy phDC.

In one embodiment, the dendritic cells of step a) have been obtained by plate passage of PBMC from the subject. In one embodiment, the dendritic cells of step c) have been obtained by plate passage of PBMC from the subject. Thus, the dendritic cells of step a) and/or step c) can be termed physiologic DC. All embodiments relating to the generation of phDC as described for the first aspect also apply to the generation of phDC for the tenth aspect. In one embodiment, the dendritic cells of step a) have been obtained from an extracorporeal blood sample which has been obtained from the subject. In one embodiment, the dendritic cells of step a) have been obtained from an extracorporeal blood sample which has been obtained from the subject and the dendritic cells of step c) have been obtained by plate passage of PBMC from the subject.

Thus, in an especially preferred embodiment, phDC of the subject are obtained by subjecting monocytes contained in a blood sample to a shear force by passing the blood sample or fraction thereof through a flow chamber of a device. Preferably, platelets are present in the flow chamber which can be either derived from the subject's blood sample or a fraction thereof or provided separately. Additionally or alternatively, plasma components can be present in the flow chamber which can be either derived from the subject's blood sample or a fraction thereof or provided separately. However, the generation of phDC also works in the absence of platelets and/or plasma components.

A monocyte of a subject may be obtained by any suitable means, e.g., from a blood sample or a fraction thereof. The fraction of the blood sample may be, e.g., a buffy coat including white blood cells and platelets. Alternatively, the fraction of the blood sample may be an isolated peripheral blood mononuclear cell (PBMC). PBMCs may be isolated from a blood sample using, e.g., centrifugation over a Ficoll-Hypaque gradient (Isolymph, CTL Scientific). In another example, the fraction of the blood sample may be a purified or enriched monocyte preparation. Monocytes may be enriched from PBMCs using, e.g., one, two, or all three of plastic adherence; CD14 magnetic bead positive selection (e.g., from Miltenyi Biotec); and a Monocyte Isolation Kit II (Miltenyi Biotec).

Any suitable volume of blood can be used. The blood sample (e.g., the blood sample from which the fraction is derived) may be between about 1 μL and about 500 mL, e.g., between about 1 μL and about 10 mL, between about 1 μL and about 5 mL, between about 1 μL and about 1 mL, between about 1 μL and about 750 μL, between about 1 μL and about 500 μL, between about 1 μL and about 250 μL, between about 10 mL and about 450 mL, about 20 mL and about 400 mL, about 30 mL and about 350 mL, about 40 mL and about 300 mL, about 50 mL and about 200 mL, or about 50 mL and about 100 mL. In some embodiments, the blood sample or the fraction thereof or the additional blood sample or the fraction thereof is less than or equal to about 100 mL (e.g., about 50 mL to about 100 mL).

In some embodiments, the ECP device is a miniaturized ECP device, e.g. a transimmunization (TI) plate. In some embodiments, the ECP device is a plastic bag.

The skilled person is well aware of methods to distinguish dendritic cells including phDC from monocytes, such as e.g. by assessing gene expression.

Without being bound to a scientific theory, the inventors assume at present that the remarkable effects of the present invention are due to damaged dying DCs from the subject, in particular damaged by the combination of a psoralen and UVA (PUVA), which provide antigens to the physiologic DCs of the subject and a tolerance signal to the immune system of the subject. If phDCs have received such tolerogenic signals from PUVA-treated apoptotic autologous DCs, the phDCs (in addition to the received tolerogenic signal) may display antigens, in particular autoantigens, from the autologous PUVA-treated apoptotic DCs on their surface and thereby trigger an antigen-specific tolerogenic response. Analogous, the source of the antigen may also be derived from immune cells, e.g. lymphocytes. Thus, immune cells, e.g. lymphocytes, may be rendered apoptotic and combined with phDCs of the subject in order to generate an antigen-specific tolerogenic response.

In one embodiment, the dendritic cells of step a) are derived from an extracorporeal blood sample of the subject. In another embodiment, the dendritic cells of step a) have been obtained by plate passage of PBMC from the subject.

In one embodiment, the invention relates to a method comprising the following steps (with all embodiments as described above also applying mutatis mutandis to the following embodiment):
a) Providing a first sample of dendritic cells obtained from a subject;
a1) Incubating the dendritic cells with antigenic molecules;
b) Exposing the incubated dendritic cells of step a1) to an apoptotic agent;
c) Providing a second sample of dendritic cells obtained from the subject; and
d) Combining the apoptotic dendritic cells of step b) with the dendritic cells of step c).

In one embodiment, the dendritic cells of step a) have been obtained by plate passage of PBMC from the subject. In one embodiment, the dendritic cells of step c) have been obtained by plate passage of PBMC from the subject. In one embodiment, the dendritic cells of step a) have been obtained from an extracorporeal blood sample from the subject. In one embodiment, the dendritic cells of step a) have been obtained from an extracorporeal blood sample from the subject and the dendritic cells of step c) have been obtained by plate passage of PBMC from the subject.

In one embodiment, the antigenic molecule is an autoantigen. In one embodiment, the autoantigen is associated with one or more autoimmune disorders. In one embodiment, the antigenic molecule is derived from a natural source, chemically synthesized or recombinantly produced. In one embodiment, the antigenic molecule is derived from a cell.

Table A below shows a list of exemplary autoantigens associated with autoimmune disease(s), as well as exemplary animal model systems that can be used to evaluate ameliorization of an autoimmune disease using tolerogenic phDC (see also Experiments 7 and 8).

TABLE A
List of Exemplary Autoantigens, Associated Autoimmune Disease(s), and Animal Model(s)

| | | Exemplary | | Exemplary |
|---|---|---|---|---|
| | Exemplary | References for | | References |
| | Autoimmune | Autoimmune | | for Animal |
| Autoantigen | Disease(s) | Disease(s) | Animal Model(s) | Models |
| Rh blood group | Autoimmune | Iwamoto et al. | | |
| antigens | hemolytic anemia | Am. J. | | |
| | | Hematol. 68.2 | | |
| | | (2001): 106- | | |
| | | 114 | | |
| Platelet integrin | Immune | Kuwana et al. | | |
| GpIIb:IIIa | thrombocytopenic | Blood 103.4 | | |
| | purpura (ITP) | (2004): 1229- | | |
| | (also known as | 1236 | | |
| | autoimmune | | | |
| | thrombocytopenic | | | |
| | purpura and | | | |
| | Idiopathic | | | |
| | thrombocytopenic | | | |
| | purpura) | | | |
| Noncollagenous | Goodpasture's | Kalluri et al. | Alpha-3NC1 | Kalluri et al. |
| domain (NC1) of | syndrome | Proc. Natl. | immunized rabbit | Proc. Natl. |
| basement | | Acad. Sci. | | Acad. Sci. |
| membrane | | U.S.A 91.13 | | U.S.A 91.13 |
| collagen type IV | | (1994): 6201- | | (1994): 6201- |
| | | 6205 | | 6205 |
| Epidermal | Vitiligo and | Vitiligo | Pemphigus: Ecad- | Wagner et al. |
| cadherin | pemphigus | (Wagner et al. | deficient | J Invest |
| | | J Invest | melanocytes | Dermatol |
| | | Dermatol | (ΔEcad) mouse | 135.7 (2015) |
| | | 135.7 (2015) | | 1810-1819 |
| | | 1810-1819); | | |
| | | Pemphigus | | |
| | | (Evangelista et | | |
| | | al. J Invest | | |
| | | Dermatol 128.7 | | |
| | | (2008): 1710- | | |
| | | 1718) | | |
| Streptococcal cell- | Streptococcal cell | van den Broek | Lewis and/or | van den Broek |
| wall antigens | wall-induced | et al. Cell. | Fischer 344 | et al. Cell. |
| | arthritis | Immunol. 116.1 | (F344) rat | Immunol. 116.1 |
| | | (1988): 216- | | (1988): 216- |
| | | 229 | | 229 |
| Rheumatoid factor | Autoimmune | Ghonaim et al. | | |
| (RF) IgG | hepatitis (AIH) | Egypt J | | |
| complexes with | | Immunol 12.2 | | |
| hepatitic C | | (2005): 101- | | |
| antigens | | 111 | | |
| Rheumatoid factor | Autoimmune | Ghonaim et al. | | |
| (RF) IgG | hepatitis (AIH) | Egypt J | | |
| complexes without | | Immunol 12.2 | | |
| hepatitic C | | (2005): 101- | | |
| (antigens | | 111 | | |
| Pancreatic β-cell | Insulin-dependent | Lieberman et | Nonobese diabetic | Lieberman et |
| antigen | diabetes mellitus | al. Proc. Natl. | (NOD) mouse | al. Proc. Natl. |
| | (IDDM) | Acad. Sci. | | Acad. Sci. |
| | | U.S.A 100.12 | | U.S.A 100.12 |
| | | (2003): 8384- | | (2003): 8384- |
| | | 8388 | | 8388 |
| Myelin basic | Multiple sclerosis | Reindl et al. | Experimental | Lafeaille et |
| protein (MBP) | (MS) | Brain 122.11 | autoimmune | al. Cell 78.3 |
| | | (1999): 2047- | encephalomyelitis | (1994): 399- |
| | | 2056 | as model for MS | 408 |
| | | | induced by | |
| | | | T cell receptor | |
| | | | (TCR) transgenic | |
| | | | mouse specific for | |
| | | | MBP and crossed | |
| | | | to RAG-1- | |
| | | | deficient mouse to | |
| | | | obtain mouse | |
| | | | (TR−) that have T | |
| | | | cells expressing | |
| | | | the transgenic | |
| | | | TCR but no other | |
| | | | lymphocytes | |
| Proteolipid protein | Multiple sclerosis | Sun et al. Eur. | PLP immunized | Terry et al. |
| (PLP) | (MS) | J. Immunol. | mouse (elicit | Mult. Scler. |
| | | 21.6 (1991): | experimental | 1304 (2016): |
| | | 1461-1468 | autoimmune | 145-160 |
| | | | encephalomyelitis | |
| | | | as MS model) | |
| Myelin | Multiple sclerosis | Bernard et al. J. | MOG immunized | MOG (Terry |
| oligodendrocyte | (MS) | Mol. Med 75.2 | mouse or IL-6- | et al. Mult. |
| glycoprotein | | (1997): 77-78 | deficient mouse | Scler. 1304 |
| (MOG) | | | (both elicit | (2016): 145- |
| | | | experimental | 160); IL-6 |
| | | | autoimmune | (Okuda et al. |
| | | | encephalomyelitis | Int. Immunol. |
| | | | as MS model) | 10.5 (1998): |
| | | | | 703-708 |
| Desmoglein 3 | Paraneoplastic | Amagai et al. J. | Paraneoplastic | Amagai et al. J. |
| | pemphigus | Clin. Investig | pemphigus | Clin. Investig |
| | | 102.4 (1998): | immunized mouse | 102.4 (1998): |
| | | 775-782 | | 775-782 |
| Glutamic acid | Insulin-dependent | Maclaren et al. | | |
| decarboxylase | diabetes mellitus | J. Autoimmun | | |
| | (IDDM) | 12.4 (1999): | | |
| | | 279-287 | | |
| Acetylcholine | Autoimmune | Autoimmune | | |
| receptor | autonomic | autonomic | | |
| | ganglionopathy | ganglionopathy | | |
| | and Sjogren's | (Vernino et al. | | |
| | syndrome | J. | | |
| | | Neuroimmunol | | |
| | | 197.1 (2008): | | |
| | | 63-69), | | |
| | | Sjogren's | | |
| | | syndrome | | |
| | | (Naito et al. | | |
| | | Ann. Rheum. | | |
| | | 64.3 (2005): | | |
| | | 510-511) | | |
| Carboxypeptidase | Insulin-dependent | Castano et al. J. | | |
| H | diabetes mellitus | Clin. | | |
| | (IDDM) | Endocrinol. | | |
| | | Metab. 73.6 | | |
| | | (1991): 1197- | | |
| | | 1201 | | |
| Chromogranin A | Insulin-dependent | Gottlieb et al. J. | Nonobese diabetic | Baker et al. |
| | diabetes mellitus | Autoimmun 50 | (NOD) mouse | J. Immunol. |
| | (IDDM) | (2014): 38-41 | | 196.1 (2016): |
| | | | | 39-43 |
| Glutamate | Insulin-dependent | Myers et al. | | |
| decarboxylase | diabetes mellitus | Diabetes 44.11 | | |
| | (IDDM) | (1995): 1290- | | |
| | | 1295; Maclaren | | |
| | | et al. J. | | |
| | | Autoimmun | | |
| | | 12.4 (1999): | | |
| | | 279-287 | | |
| Imogen-38 | Insulin-dependent | Arden et al. J. | | |
| | diabetes mellitus | Clin. Investig. | | |
| | (IDDM) | 97.2 (1996): | | |
| | | 551-561 | | |
| Insulin | Insulin-dependent | Maclaren et al. | Nonobese diabetic | Nakayama et |
| | diabetes mellitus | J. Autoimmun | (NOD) mouse | al. Nature |
| | (IDDM) | 12.4 (1999): | | 435.7039 |
| | | 279-287 | | (2005): 220- |
| | | | | 223 |
| Insulinoma | Insulin-dependent | Maclaren et al. | | |
| antigen-2 | diabetes mellitus | J. Autoimmun | | |
| | (IDDM) | 12.4 (1999): | | |
| | | 279-287 | | |
| Insulinoma antigen | Insulin-dependent | Maclaren et al. | | |
| 2β | diabetes mellitus | J. Autoimmun | | |
| | (IDDM) | 12.4 (1999): | | |
| | | 279-287 | | |
| Islet-specific | Insulin-dependent | Wong et al. J. | Nonobese diabetic | Wong et al. J. |
| glucose-6- | diabetes mellitus | Immunol. 176.3 | (NOD) mouse | Immunol. 176.3 |
| phosphatase | (IDDM) | (2006): 1637- | | (2006): 1637- |
| catalytic subunit | | 1644 | | 1644 |
| related protein | | | | |
| (IGRP) | | | | |
| Proinsulin | Insulin-dependent | Michels et al. | | |
| | diabetes mellitus | Diabetes 66.3 | | |
| | (IDDM) | (2017): 722- | | |
| | | 734 | | |
| A-enolase | Autoimmune- | Magrys et al. J. | | |
| | associated | Clin. Immunol. | | |
| | retinopathy | 27.2 (2007): | | |
| | | 181-192 | | |
| Aquaporin-4 | Multiple sclerosis | Mirshafiey et | Neuromyelitis | Jones et al. |
| | (MS) and | al. Iran J | optica: AQP4 null | Acta |
| | neuromyelitis | Allergy Asthma | mouseimmunized | Neuropathol. |
| | optica | Immunol 12.4 | with AQP4 | Commun 3.1 |
| | | (2013): 292- | | (2015): 1-8 |
| | | 303 | | |
| β-arrestin | Multiple sclerosis | Ohguro et al. | | |
| | (MS) | Proc. Natl. | | |
| | | Acad. Sci. | | |
| | | U.S.A 90.8 | | |
| | | (1993): 3241- | | |
| | | 3245 | | |
| S100-β | Insulin-dependent | Calvino- | | |
| | diabetes mellitus | Sampedro et al. | | |
| | (IDDM) | FASEB J 33.5 | | |
| | | (2019): 6390- | | |
| | | 6401 | | |
| Citrullinated | Rheumatoid | Sokolove et al. | | |
| protein | arthritis (RA) | Arthritis | | |
| | | Rheumatol. | | |
| | | 66.4 (2014): | | |
| | | 813-821 | | |
| Collagen II | Collagen-induced | Stuart et al. | Collagen II | Stuart et al. |
| | arthritis | Annu. Rev. | immunized rat | Annu. Rev. |
| | | Immunol 2.1 | | Immunol 2.1 |
| | | (1984): 199- | | (1984): 199- |
| | | 218 | | 218 |
| Heat shock | Autoimmune | Autoimmune | Glaucoma: HSP70 | Casola et al. |
| proteins | pancreatitis, | pancreatitis and | immunized rats | J. Mol. |
| | insulin-dependent | IDMM | | Neurosci. |
| | diabetes mellitus | (Takizawa et al. | | 56.1 (2015): |
| | (IDDM) and | Biochem. | | 228-236 |
| | glaucoma | Biophys. Res. | | |
| | | Commun. 386.1 | | |
| | | (2009): 192- | | |
| | | 196); glaucoma | | |
| | | (Wakefield & | | |
| | | Wildner Clin | | |
| | | Transl | | |
| | | Immunology | | |
| | | 9.10 (2020): | | |
| | | e1180) | | |
| Human cartilage | Rheumatoid | Hakala and | HC gp-39 | Verheijden et |
| glycoprotein 39 | arthritis (RA) | Recklies., J. | immunized mouse | al. Arthritis |
| (HC gp-39) | | Biol. Chem | | Rheum 40.6 |
| | | 268.34 (1993): | | (1997): |
| | | 25803-25810 | | 1115-1125 |
| La antigen (MAM: | Systemic Lupus | Arvieux et al. | | |
| assuming this | Erythematosus | Thromb | | |
| means lupus | (SLE) | Haemost 74.10 | | |
| anticoagulants) | | (1995): 1120- | | |
| | | 1125 | | |
| Nucleosomal | Systemic Lupus | Chabre et al. | | |
| histones | Erythematosus | Arthritis | | |
| | (SLE) | Rheumatol. | | |
| | | 38.10 (1995): | | |
| | | 1485-1491 | | |
| Nucleosomal | Systemic Lupus | Mamula et al., | | |
| ribonucleoproteins | Erythematosus | J. Immunol. | | |
| (snrnp) | (SLE) | 152.3 (1994): | | |
| | | 1453-1461 | | |
| Phospholipid-β-2 | Systemic Lupus | Arvieux et al. | | |
| glycoprotein I | Erythematosus | Thromb | | |
| complex | (SLE) | Haemost 74.10 | | |
| | | (1995): 1120- | | |
| | | 1125 | | |
| Poly(ADP-ribose) | Systemic Lupus | Kanai et al., | MRL/Mp-lpr/lpr | Kanai et al., |
| polymerase | Erythematosus | Proc Jpn Acad | mouse | Clin. Exp. |
| | (SLE) | Ser B Phys Biol | | Immunol. |
| | | Sci. 92.7 | | 59.1 (1985): |
| | | (2016): 222- | | 132 |
| | | 236 | | |
| Sm antigens of U- | Systemic Lupus | Lerner et al. | MRL/Mp-lpr/lpr | Lerner et al. |
| 1 small | Erythematosus | Proc. Natl. | mouse | Proc. Natl. |
| ribonucleoprotein | (SLE) | Acad. Sci. | | Acad. Sci. |
| complex | | U.S.A 78.5 | | U.S.A 78.5 |
| | | (1981): 2737- | | (1981): |
| | | 2741 | | 2737-2741 |
| Pancreatic islet | Insulin-dependent | MacCuish et al. | | |
| cell antigens | diabetes mellitus | Lancet | | |
| | (IDDM) | 304.7896 | | |
| | | (1974): 1529- | | |
| | | 1531 | | |
| Cytoplasmic linker | Systemic Lupus | Griffith et al. | | |
| protein-170 | Erythematosus | Clin. Exp. | | |
| (CLIP-170) | (SLE) and limited | Immunol. 127.3 | | |
| | cutaneous | (2002): 533- | | |
| | systemic sclerosis | 538 | | |
| Sjogren's | Neonatal lupus | Yukiko et al. | | |
| syndrome antigen | erythematosus | Br. J. | | |
| A (SS-A/Ro) | (NLE) | Dermatol. | | |
| | | 142.5 (2000): | | |
| | | 908-912 | | |
| Sjogren's | Neonatal lupus | Yukiko et al. | | |
| syndrome antigen | erythematosus | Br. J. | | |
| B (SS-B/La) | (NLE) | Dermatol. | | |
| | | 142.5 (2000): | | |
| | | 908-912 | | |
| Sjogren's lupus | Sjogren's | Francoeur et al. | | |
| antigen (SL) | syndrome and | Mol Cell Biol | | |
| | Systemic Lupus | 5.3 (1985): | | |
| | Erythematosus | 586-590 | | |
| | (SLE) | | | |
| Scleroderma | Scleroderma, | Guldner et al. | | |
| antigen 70 (Scl-70) | progressive | Chromosoma | | |
| | systemic sclerosis | 94.2 (1986): | | |
| | | 132-138 | | |

In one embodiment, the autoantigen is selected from the group comprising Rh blood group antigens, platelet integrin GpIIb:IIIa, noncollagenous domain of basement membrane collagen type IV, epidermal cadherin, streptococcal cell-wall antigens, rheumatoid factor IgG complexes with or without hepatitic C antigens, pancreatic β-cell antigen, myelin basic protein, proteolipid protein, myelin oligodendrocyte glycoprotein, desmoglein 3, glutamic acid decarboxylase, acetylcholine receptor, carboxypeptidase H, chromogranin A, glutamate decarboxylase, imogen-38, insulin, insulinoma antigen-2 and 2β, islet-specific glucose-6-phosphatase catalytic subunit related protein (IGRP), proinsulin, α-enolase, aquaporin-4, β-arrestin, S100-β, citrullinated protein, collagen II, heat shock proteins, human cartilage glycoprotein 39, La antigen, nucleosomal histones and ribonucleoproteins (snRNP), phospholipid-β-2 glycoprotein I complex, poly (ADP-ribose) polymerase, Sm antigens of U-1 small ribonucleoprotein complex, pancreatic islet cell antigens, cytoplasmic linker protein-170 (CLIP-170), Sjogren's syndrome antigen A (SS-A/Ro), Sjogren's syndrome antigen B (SS-B/La), Sjogren's lupus antigen (SL) and scleroderma antigen 70 (Scl-70)). In one embodiment, the autoantigen is selected from myelin basic protein and collagen.

In one embodiment, the subject is mammalian. Mammals include for example, but are not limited to, humans, non-human primates, pigs, dogs, cats, horses and rodents. In a preferred embodiment, the subject is human.

All of the above embodiments may be carried out in vitro.

The methods of the present invention can be applied in conjunction with other therapies for the treatment of autoimmune diseases. For example, the autoimmune disease may be any autoimmune disease described in Table A above. Other autoimmune diseases are known in the art.

### Eleventh Aspect: Tolerogenic Dendritic Cells Obtained by a Method According to the Tenth Aspect

In an eleventh aspect, the present invention relates to tolerogenic dendritic cells obtained by a method according to the tenth aspect (including all embodiments as described above).

### Twelfth Aspect: Tolerogenic Dendritic Cells According to the Eleventh Aspect for Use in the Treatment of Autoimmune Diseases

In a twelfth aspect, the present invention relates to tolerogenic dendritic cells according to the eleventh aspect (including all embodiments of the tenth and eleventh aspect as described above) for use in the treatment of autoimmune diseases.

For the treatment of autoimmune diseases, it is contemplated that administration of the tolerogenic dendritic cells will result in some ameliorative effect, such as an improvement in the quality of life; a reduction in the severity of the symptoms of the disease; a reduction in the number of autoimmune cells; prolonged survival, and the like.

Indicators of beneficial effect are well known in the art, and an appropriate indicator for a particular application can be determined by the skilled person.

Following such a treatment, the severity of the autoimmune disease or symptoms associated therewith, is significantly reduced compared to not administering the tolerogenic dendritic cells.

In one embodiment, the autoimmune disease is selected from the group comprising multiple sclerosis, rheumatoid arthritis, juvenile rheumatoid arthritis, systemic lupus erythematosus, amyotrophic lateral sclerosis; pemphigus vulgaris, psoriasis, myasthenia gravis, thyroiditis, scleroderma, Sjogren's syndrome, thrombocytopenic purpura, cryoglobulinemia, autoimmune haemolytic anemia, insulin-dependent diabetes mellitus (IDDM), Addison's disease, celiac disease, chronic fatigue syndrome, colitis, Crohn's disease, fibromyalgia, hyperthyroidism/Graves disease, hypothyroidism/Hashimoto's disease, endometriosis, pernicious anemia, Goodpasture syndrome, Wegener's disease and rheumatic fever.

In another embodiment, provided herein is a method of treating an autoimmune disease in a subject in need thereof, the method comprising administering an effective amount of any of the tolerogenic dendritic cells described herein to the subject.

In another embodiment, provided herein is a method of treating an autoimmune disease in a subject in need thereof, the method comprising administering an effective amount of tolerogenic dendritic cells to the subject, wherein the tolerogenic dendritic cells comprise physiological dendritic cells comprising material from an apoptotic dendritic cell obtained from the subject, an autoantigen, a fragment thereof, or a combination thereof.

In any of the preceding methods, the autoimmune disease may be selected from the group comprising multiple sclerosis, rheumatoid arthritis, juvenile rheumatoid arthritis, systemic lupus erythematosus, amyotrophic lateral sclerosis; pemphigus vulgaris, psoriasis, myasthenia gravis, thyroiditis, scleroderma, Sjogren's syndrome, thrombocytopenia purpura, cryoglobulinemia, autoimmune haemolytic anemia, insulin-dependent diabetes mellitus (IDDM), Addison's disease, celiac disease, chronic fatigue syndrome, colitis, Crohn's disease, fibromyalgia, hyperthyroidism/Graves disease, hypothyroidism/Hashimoto's disease, endometriosis, pernicious anemia, Goodpasture syndrome, Wegener's disease and rheumatic fever.

In any of the preceding methods, the autoantigen may be selected from the group comprising Rh blood group antigens, platelet integrin GpIIb:IIIa, noncollagenous domain of basement membrane collagen type IV, epidermal cadherin, streptococcal cell-wall antigens, rheumatoid factor IgG complexes with or without hepatitic C antigens, pancreatic β-cell antigen, myelin basic protein, proteolipid protein, myelin oligodendrocyte glycoprotein, desmoglein 3, glutamic acid decarboxylase, acetylcholine receptor, carboxypeptidase H, chromogranin A, glutamate decarboxylase, imogen-38, insulin, insulinoma antigen-2 and 2β, islet-specific glucose-6-phosphatase catalytic subunit related protein (IGRP), proinsulin, α-enolase, aquaporin-4, β-arrestin, S100-β, citrullinated protein, collagen II, heat shock proteins, human cartilage glycoprotein 39, La antigen, nucleosomal histones and ribonucleoproteins (snRNP), phospholipid-β-2 glycoprotein I complex, poly (ADP-ribose) polymerase, Sm antigens of U-1 small ribonucleoprotein complex, pancreatic islet cell antigens, cytoplasmic linker protein-170 (CLIP-170), Sjogren's syndrome antigen A (SS-A/Ro), Sjogren's syndrome antigen B (SS-B/La), Sjogren's lupus antigen (SL) and scleroderma antigen 70 (Scl-70)).

### Thirteenth Aspect: Ex Vivo Tolerogenic Dendritic Cells

In a thirteenth aspect, the invention relates to an ex vivo tolerogenic dendritic cell comprising material from an apoptotic dendritic cell obtained from a subject.

In some embodiments, the material from an apoptotic dendritic cell obtained from a subject includes polypeptides, nucleic acids, organelles or portions thereof, or any other cell contents.

In some embodiments, the ex vivo tolerogenic dendritic cell includes an autoantigen or a fragment thereof. Any suitable autoantigen may be included, including any autoantigen described herein (e.g., in Table A). For polypeptide antigens, the fragment may be of any suitable size, e.g., about 5, 6, 7, 8, 9, 10, 11, 12, 13, 14, 15, 16, 17, 18, 19, 20, 25, 30, 35, 40, 45, 50, 75, 100, 125, 150, 175, 200, 225, 250, 275, 300, 325, 350, 375, 400, 425, 450, 475, 500, 600, 700, 800, 900, or 1000 amino acids.

In some embodiments, the ex vivo tolerogenic dendritic cells include one type of autoantigen or a fragment thereof. In other embodiments, the ex vivo tolerogenic dendritic cells may include two, three, four, five, ten, or more different autoantigens or fragments thereof.

In some embodiments, the autoantigen is selected from the group comprising Rh blood group antigens, platelet integrin GpIIb:IIIa, noncollagenous domain of basement membrane collagen type IV, epidermal cadherin, streptococcal cell-wall antigens, rheumatoid factor IgG complexes with or without hepatitic C antigens, pancreatic β-cell antigen, myelin basic protein, proteolipid protein, myelin oligodendrocyte glycoprotein, desmoglein 3, glutamic acid decarboxylase, acetylcholine receptor, carboxypeptidase H, chromogranin A, glutamate decarboxylase, imogen-38, insulin, insulinoma antigen-2 and 2β, islet-specific glucose-6-phosphatase catalytic subunit related protein (IGRP), proinsulin, α-enolase, aquaporin-4, β-arrestin, S100-β, citrullinated protein, collagen II, heat shock proteins, human cartilage glycoprotein 39, La antigen, nucleosomal histones and ribonucleoproteins (snRNP), phospholipid-β-2 glycoprotein I complex, poly (ADP-ribose) polymerase, Sm antigens of U-1 small ribonucleoprotein complex, pancreatic islet cell antigens, cytoplasmic linker protein-170 (CLIP-170), Sjogren's syndrome antigen A (SS-A/Ro), Sjogren's syndrome antigen B (SS-B/La), Sjogren's lupus antigen (SL) and scleroderma antigen 70 (Scl-70)).

The subject may be suffering from an autoimmune disease, including any autoimmune disease disclosed herein (e.g., in Table A).

Fourteenth Aspect: Compositions that Include a Sample of Dendritic Cells Obtained from a Subject

In a fourteenth aspect, the invention relates to a composition that includes (a) a sample of dendritic cells obtained from a subject; (b) an apoptotic agent; and (c) an autoantigen or a fragment thereof.

The composition may include any suitable apoptotic agent, including any apoptotic agent disclosed herein. In some embodiments, the apoptotic agent includes a psoralen, riboflavin-phosphate, 5-aminolevulinic acid, or a combination thereof. In some embodiments, the apoptotic agent is a psoralen. In some embodiments, the psoralen is selected from the group comprising 8-MOP and amotosalen. In some embodiments, the psoralen is 8-MOP.

In some embodiments, the composition includes an autoantigen or a fragment thereof. Any suitable autoantigen may be included, including any autoantigen described herein (e.g., in Table A). For polypeptide antigens, the fragment may be of any suitable size, e.g., about 5, 6, 7, 8, 9, 10, 11, 12, 13, 14, 15, 16, 17, 18, 19, 20, 25, 30, 35, 40, 45, 50, 75, 100, 125, 150, 175, 200, 225, 250, 275, 300, 325, 350, 375, 400, 425, 450, 475, 500, 600, 700, 800, 900, or 1000 amino acids.

In some embodiments, the composition includes one type of autoantigen or a fragment thereof. In other embodiments, the composition may include two, three, four, five, ten, or more different autoantigens or fragments thereof.

In some embodiments, the autoantigen is selected from the group comprising Rh blood group antigens, platelet integrin GpIIb:IIIa, noncollagenous domain of basement membrane collagen type IV, epidermal cadherin, streptococcal cell-wall antigens, rheumatoid factor IgG complexes with or without hepatitic C antigens, pancreatic β-cell antigen, myelin basic protein, proteolipid protein, myelin oligodendrocyte glycoprotein, desmoglein 3, glutamic acid decarboxylase, acetylcholine receptor, carboxypeptidase H, chromogranin A, glutamate decarboxylase, imogen-38, insulin, insulinoma antigen-2 and 2β, islet-specific glucose-6-phosphatase catalytic subunit related protein (IGRP), proinsulin, α-enolase, aquaporin-4, β-arrestin, S100-β, citrullinated protein, collagen II, heat shock proteins, human cartilage glycoprotein 39, La antigen, nucleosomal histones and ribonucleoproteins (snRNP), phospholipid-β-2 glycoprotein I complex, poly (ADP-ribose) polymerase, Sm antigens of U-1 small ribonucleoprotein complex, pancreatic islet cell antigens, cytoplasmic linker protein-170 (CLIP-170), Sjogren's syndrome antigen A (SS-A/Ro), Sjogren's syndrome antigen B (SS-B/La), Sjogren's lupus antigen (SL) and scleroderma antigen 70 (Scl-70)).

The dendritic cells may be obtained from a subject suffering from an autoimmune disease, including any autoimmune disease disclosed herein (e.g., in Table A).

### FIGURE LEGENDS

FIG. 1 Schematic design of ECP leukemia haploidentical trial.

FIG. 2 Schematic design of ECP leukemia haploidentical trial.

FIG. 3 Schematic design of Balb/C→B6 full mismatch GVHD system; results are also depicted.

FIG. 4 Ex vivo psoralen UVA treatment (PUVA) of graft ameliorates GVHD in a full MHC mismatch model. Mice were injected s.c. with 2×10⁵ MC38 tumor cells before transplant on day −4 or the day of transplant on day 0. Mice were lethally irradiated at 950 cGy on day −1. On day 0, they underwent Balb/c→B6 transplant. They received i.v. injections of 5×10⁶ allogeneic T cell depleted bone marrow (BM) cells along with 10×10⁶ splenocytes unmanipulated, or following ex vivo PUVA treatment of the allo-stimulated graft. As controls, a group of mice received syngeneic BM and splenocytes following tumor inoculation. (A, panel 1-3) Pooled data of average weight, GvHD score and survival of all groups. (B) Average tumor volume.

FIG. 5 Cardiac transplant Kaplan-Meier's Survival Curve.

FIG. 6 Subject characteristics.

FIG. 7 GVHD grade and stage.

FIG. 8 Cumulative incidence of acute GVHD. Unrelated donor analysis includes one patient who had a 5/6 HLA-matched related donor.

FIG. 9 Cumulative incidence of extensive chronic GVHD.

FIG. 10 Overall survival. Unrelated donor analysis includes one patient who had a 5/6 HLA-matched related donor.

FIG. 11 Cumulative incidence of transplant-related mortality. Unrelated donor analysis includes one patient who had a 5/6 HLA-matched related donor.

FIG. 12 Selection criteria for historical controls.

FIG. 13 Characteristics of study and historical control subjects.

FIG. 14 Relative risk and 95% confidence interval of transplant outcomes in multivariate analysis (the control group is used as a reference and assigned a relative risk of 1.0).

FIG. 15 Cumulative incidence of grades II-IV acute GVHD.

FIG. 16 Adjusted probability of disease-free survival.

FIG. 17 Adjusted probability of survival.

FIG. 18 PD-L1 expression of fresh PBMC alone versus phDC which have been incubated with 8-MOP/UVA damaged syngeneic PBMC (PUVA syn PBMC) or 8-MOP/UVA damaged allogeneic PBMC (PUVA allo PBMC).

FIG. 19 CFSE-labeled responder T cells from one donor (T cells) were co-incubated with gamma-irradiated stimulator PBMC either from the same donor (syn. culture) or from an unrelated donor (MLR). In order to suppress the MLR reaction, some cultures were additionally supplemented with syngeneic 8-MOP/UVA-treated PBMC, and syngeneic TI plate-passed phDC (MLR+PUVA syn. PBMC+phDC). Proliferation of responder CD8 and CD4 T cells was assayed by measuring CFSE dilution by flow cytometry (FACS) (A, B). The activation state of responder CD8 and CD4 T cells was additionally assessed by FACS, using CD44 and PD1 expression to detect activated T cells (C, D). N=number of blood donors analyzed; p-value=unpaired t test with Welch's correction.

### DETAILED DESCRIPTION OF THE INVENTION

The following general definitions are provided.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group, which preferably consists only of these embodiments.

For the purposes of the present invention, the term “obtained” is considered to be a preferred embodiment of the term “obtainable”. If hereinafter e.g. an antibody is defined to be obtainable from a specific source, this is also to be understood to disclose an antibody, which is obtained from this source.

Where an indefinite or definite article is used when referring to a singular noun, e.g. “a”, “an” or “the”, this includes a plural of that noun unless something else is specifically stated. The terms “about” or “approximately” in the context of the present invention denote an interval of accuracy that the person skilled in the art will understand to still ensure the technical effect of the feature in question. The term typically indicates deviation from the indicated numerical value of ±20%, preferably ±15%, more preferably ±10%, and even more preferably ±5%.

Furthermore, the terms “first”, “second”, “third” or “(a)”, “(b)”, “(c)”, “(d)” or “(i)”, “(ii)”, “(iii)”, “(iv)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

In case the terms “first”, “second”, “third” or “(a)”, “(b)”, “(c)”, “(d)” or “(i)”, “(ii)”, “(iii)”, “(iv)” etc. relate to steps of a method or use or assay there is no time or time interval coherence between the steps unless indicated otherwise, i.e. the steps may be carried out simultaneously or there may be time intervals of seconds, minutes, hours, days, weeks, months or even years between such steps, unless otherwise indicated in the application as set forth herein above or below.

Technical terms are used by their common sense. If a specific meaning is conveyed to certain terms, definitions of terms will be given in the following in the context of which the terms are used.

As used herein, “transplant” refers to any sample of cells that is removed from a mammalian individual (a “donor”) and is suitable to be reintroduced, in whole or in part, into the same (“autologous”) or different (“allogeneic”) mammalian individual (a “recipient”). The transplant can be either freshly obtained, cultured or frozen, but has been maintained under conditions suitable to maintain sterility and promote viability. The term transplant is used interchangeably with the term graft.

The methods of the invention can be practiced with individuals who are closely HLA-matched, sharing all or nearly all of their class I and class II HLA antigens; haploidentical, such as siblings sharing half of their HLA antigens; or unrelated, and thus poorly HLA matched. In the context of the present invention “haplo-donor” refers to one genetic parent of a future recipient while the term “complimentary haplo-donor” refers to the other genetic parent. The future recipient is thus the child. Put in other words, if the mother is the haplo-donor, the father is the complimentary haplo-donor of the child (future recipient) and vice versa. In a preferred embodiment of the first aspect, the recipient and donor are unrelated. In a preferred embodiment of the second aspect, the haplo-donor and complimentary haplo-donor each have half of the future recipient's HLA antigens.

The degree of HLA identity between individuals can readily be demonstrated by methods known in the art, including the polymerase chain reaction, mixed lymphocyte reactions (MLR), and serological measurements.

As used herein, the term “antigen” refers to a compound, composition, or substance that can stimulate the production of antibodies or a T cell response in an animal, including compositions that are injected or absorbed into an animal. An antigen reacts with the products of specific humoral or cellular immunity, including those induced by heterologous immunogens. The term is used interchangeably with the term “immunogen” or “antigenic molecule”. The term “antigen” includes all related antigenic epitopes. The terms “antigen”, “antigenic molecule” or “immunogen” include fragments thereof that are still capable of acting as an antigen. “Epitope” or “antigenic determinant” refers to a site on an antigen to which B and/or T cells respond. In one example, the recipient antigen includes antigens from dendritic cells, such as dendritic cells obtained from plate passage of peripheral blood leukocytes (including monocytes or monocyte-derived cells).

As used herein, the term “immunogenicity” refers to the ability of a substance, a cell or a part thereof, such as an antigen, to provoke an immune response in the body of a human or animal.

As used herein, the term “autoantigen” refers to a host antigen (or microbial superantigen) considered by those skilled in the art to be associated with an autoimmune disease, such that the presence of activated T cells specific for the autoantigen is correlated with development or progression of the disease.

Autoantigens may be defined autoimmune target antigens e.g., defined autoimmune target antigens for example, in multiple sclerosis, the target antigen identified as myelin basic protein (MBP) MBP 84-102, or MBP 143-168; pancreatic islet cell antigens; in uveitis, the S Antigen; or in rheumatoid arthritis, type II or other types of collagen; in SLE, cytoplasmic linker protein-170 (CLIP-170); Sjogren's syndrome antigen A (SS-A/Ro), Sjogren's syndrome antigen B (SS-B/La), Sjogren's lupus antigen (SL); scleroderma antigen 70 (Scl-70); in Grave's disease, thyroid receptor; in Myasthena gravis, acetylcholine receptor. Autoantigens of the present invention also comprise peptide mixtures eluted from MHC molecules known to be associated with autoimmunity, for example, HLA-DQ and -DR molecules that confer susceptibility to several common autoimmune diseases, such as type 1 diabetes, rheumatoid arthritis and multiple sclerosis, or HLA-B27 molecules known to confer susceptibility to reactive arthritis and ankylosing spondylitis. Autoantigens of the present invention may also be synthesized peptides predicted to bind to WIC molecules associated with autoimmune diseases. For other autoimmune diseases, in which individual autoantigens have not yet been characterized, an autoantigen suitable for practice of the methods of the invention can be cells or a cell extract from the affected tissue (e.g. synovial cells for rheumatoid arthritis, a skin lesion for psoriasis, etc.). The term autoantigen also includes fragments thereof which act as autoantigens.

“Immune cell,” as used herein, refers broadly to cells that are of hematopoietic origin and that play a role in the immune response. Immune cells include lymphocytes, such as B cells and T cells; leukocytes; natural killer cells; and myeloid cells, such as monocytes, dendritic cells, macrophages, eosinophils, mast cells, basophils, and granulocytes.

“Dendritic cells,” also referred to herein as “DCs,” are antigen-presenting immune cells that process antigenic material and present it to other cells of the immune system, most notably to T cells. DCs function to capture and process antigens. When DCs endocytose antigens, they process the antigens into smaller fragments, generally peptides, that are displayed on the DC surface, where they are presented to, for example, antigen-specific T cells through MHC molecules. After uptake of antigens, DCs migrate to the lymph nodes. During maturation, DCs can be prompted by various signals, including signaling through Toll-like receptors (TLR), to express co-stimulatory signals that induce cognate effector T cells (T_{eff}) to become activated and to proliferate, thereby initiating a T-cell mediated immune response to the antigen. Alternatively, DCs can present an antigen to antigen-specific T cells without providing co-stimulatory signals (or while providing co-inhibitory signals), such that T_{eff} are not properly activated. Such presentation can cause, for example, death or anergy of T cells recognizing the antigen, or can induce the generation and/or expansion of regulatory T cells (T_{reg}). The term “dendritic cells” includes differentiated dendritic cells, immature, and mature dendritic cells. These cells can be characterized by expression of certain cell surface markers (e.g., CD11c, MHC class II, and at least low levels of CD80 and CD86), CD11b, CD304 (BDCA4)). In some embodiments, DCs express CD8, CD103, CD1d, etc. Other DCs can be identified by the absence of lineage markers such as CD3, CD14, CD19, CD56, etc. In addition, dendritic cells can be characterized functionally by their capacity to stimulate alloresponses and mixed lymphocyte reactions (MLR).

“Tolerogenic DCs” refers to dendritic cells capable of suppressing immune responses or generating tolerogenic immune responses, such as antigen-specific T cell-mediated immune responses, e.g., by reducing effector T cell responses to specific antigens, by effecting an increase in the number of antigen-specific regulatory T cells, etc. Tolerogenic DCs can be characterized by antigen specific tolerogenic immune response induction ex vivo and/or in vivo. Such induction refers to an induction of tolerogenic immune responses to one or more antigens of interest presented by the induced tolerogenic dendritic cells. Tolerogenic dendritic cells have a tolerogenic phenotype that may be characterized by at least one of the following properties i) capable of converting naïve T cells to Foxp3+ T regulatory cells ex vivo and/or in vivo (e.g., inducing expression of FoxP3 in the naïve T cells); ii) capable of deleting effector T cells ex vivo and/or in vivo; iii) retain their tolerogenic phenotype upon stimulation with at least one TLR agonist ex vivo (and, in some embodiments, increase expression of costimulatory molecules in response to such stimulus); iv) do not transiently increase their oxygen consumption rate upon stimulation with at least one TLR agonist ex vivo; v) increased expression of the expression marker PDL1 and/or vi) increased expression of the expression marker GILZ. Items v) and vi) may be assessed by comparison to monocytes or PBMCs.

“Tolerogenic immune response” means any immune response that can lead to immune suppression specific to an antigen or a cell, tissue, organ, etc. that expresses such an antigen. Such immune responses include any reduction, delay or inhibition in an undesired immune response specific to the antigen or cell, tissue, organ, etc. that expresses such antigen. Such immune responses also include any stimulation, production, induction, promotion or recruitment in a desired immune response specific to the antigen or cell, tissue, organ, etc. that expresses such antigen. Tolerogenic immune responses, therefore, include the absence of or reduction in an undesired immune response to an antigen that can be mediated by antigen reactive cells as well as the presence or promotion of suppressive cells. Tolerogenic immune responses as provided herein include immunological tolerance. To “generate a tolerogenic immune response” refers to the generation of any of the foregoing immune responses specific to an antigen or cell, tissue, organ, etc. that expresses such antigen.

Tolerogenic immune responses include any reduction, delay or inhibition in CD4+ T cell, CD8+ T cell or B cell proliferation and/or activity. Tolerogenic immune responses also include a reduction in antigen-specific antibody production. Tolerogenic immune responses can also include any response that leads to the stimulation, induction, production or recruitment of regulatory cells, such as CD4+ T_{reg} cells, CD8+ T_{reg} cells, B_{reg} cells, etc. In some embodiments, the tolerogenic immune response, is one that results in the conversion to a regulatory phenotype characterized by the production, induction, stimulation or recruitment of regulatory cells.

Tolerogenic immune responses also include any response that leads to the stimulation, production or recruitment of CD4+ T_{reg} cells and/or CD8+ T_{reg} cells. CD4+ T_{reg} cells can express the transcription factor FoxP3 and inhibit inflammatory responses and auto-immune inflammatory diseases (Human regulatory T cells in autoimmune diseases. Cvetanovich G L, Hafler D A. Curr Opin Immunol. 2010 December; 22 (6): 753-60. Regulatory T cells and autoimmunity. Vila J, Isaacs J D, Anderson A E. Curr Opin Hematol. 2009 July; 16 (4): 274-9). Such cells also suppress T-cell help to B-cells and induce tolerance to both self and foreign antigens (Therapeutic approaches to allergy and autoimmunity based on FoxP3+ regulatory T-cell activation and expansion. Miyara M, Wing K, Sakaguchi S. J Allergy Clin Immunol. 2009 April; 123 (4): 749-55). CD4+ T_{reg} cells recognize antigen when presented by Class II proteins on APCs. CD8+ T_{reg} cells, which recognize antigen presented by Class I, can also suppress T-cell help to B-cells and result in activation of antigen-specific suppression inducing tolerance to both self and foreign antigens. In some embodiments, the tolerogenic dendritic cells provided can effectively result in both types of responses (CD4+ T_{reg} and CD8+ T_{reg}). In other embodiments, FoxP3 can be induced in other immune cells, such as macrophages, iNKT cells, etc., the tolerogenic dendritic cells provided herein can result in one or more of these responses as well.

Tolerogenic immune responses also include, but are not limited to, the induction of regulatory cytokines, such as T_{reg} cytokines; induction of inhibitory cytokines; the inhibition of inflammatory cytokines (e.g., IL-4, IL-1b, IL-5, TNF-α, IL-6, GM-CSF, IFN-γ, IL-2, IL-9, IL-12, IL-17, IL-18, IL-21, IL-22, IL-23, M-CSF, C reactive protein, acute phase protein), chemokines (e.g., CCL-2, CXCL8, MCP-1, RANTES, MIP-1α, MIP-1β, MIG, ITAC or IP-10), the production of anti-inflammatory cytokines (e.g., IL-4, IL-13, IL-10, etc.), proteases (e.g., MMP-3, MMP-9), leukotrienes (e.g., CysLT-1, CysLT-2), prostaglandins (e.g., PGE2) or histamines; the inhibition of polarization to a Th17, Th1 or Th2 immune response; the inhibition of effector cell-specific cytokines: Th17 (e.g., IL-17, IL-25), Th1 (IFN-γ), Th2 (e.g., IL-4, IL-13); the inhibition of Th1-, Th2- or Th17-specific transcription factors; the inhibition of proliferation of effector T cells; the induction of apoptosis of effector T cells; the induction of tolerogenic dendritic cell-specific genes; the induction of FoxP3 expression; the inhibition of IgE induction or IgE-mediated immune responses; the inhibition of antibody responses (e.g., antigen-specific antibody production); the inhibition of T helper cell response; the production of TGF-β and/or IL-10; the inhibition of effector function of autoantibodies (e.g., inhibition in the depletion of cells, cell or tissue damage or complement activation); etc.

Any of the foregoing may be measured in vivo in one or more animal models or may be measured in vitro. One of ordinary skill in the art is familiar with such in vivo or in vitro measurements. Undesired immune responses or tolerogenic immune responses can be monitored using, for example, methods of assessing immune cell number and/or function, tetramer analysis, ELISPOT, flow cytometry-based analysis of cytokine expression, cytokine secretion, cytokine expression profiling, gene expression profiling, protein expression profiling, analysis of cell surface markers, PCR-based detection of immune cell receptor gene usage (see T. Clay et al., “Assays for Monitoring Cellular Immune Response to Active Immunotherapy of Cancer” Clinical Cancer Research 7:1127-1135 (2001)), etc. Undesired immune responses or tolerogenic immune responses may also be monitored using, for example, methods of assessing protein levels in plasma or serum, T cell or B cell proliferation and functional assays, etc. In some embodiments, tolerogenic immune responses can be monitored by assessing the induction of FoxP3.

Preferably, tolerogenic immune responses lead to the inhibition of the development, progression or pathology of the diseases, disorders or conditions described herein, in particular GvHD. In some embodiments, the reduction of an undesired immune response or generation of a tolerogenic immune response may be assessed by determining clinical endpoints, clinical efficacy, clinical symptoms, disease biomarkers and/or clinical scores.

As used herein, the term “animal” or “mammal” encompasses all mammals, including humans. Preferably, the mammal of the present invention is a human subject.

As used herein, the term “exposing”, refers to bringing into the state or condition of immediate proximity or direct contact.

The term “hematopoietic-cell transplantation” (HCT) is used herein to refer to blood and marrow transplantation (BMT), a procedure that involves infusion of cells (hematopoietic stem cells; also called hematopoietic progenitor cells) to reconstitute the hematopoietic system of a patient.

The term “autoimmune disorder” or “autoimmune syndrome” as used herein refers to a condition that occurs when the immune system mistakenly attacks and destroys self components of healthy body tissue. An autoimmune disorder may affect one or more organ or tissue types. Organs and tissues commonly affected by autoimmune disorders include: blood vessels, connective tissues, endocrine glands such as the thyroid or pancreas, joints, muscles, red blood cells, and skin.

In any method step of the invention that uses exposure to apoptotic agents, the apoptotic agents comprise psoralens and UVA, riboflavin-phosphate and UVA and/or aminolevulinic acid and light. Particularly preferred psoralens are 8-MOP and amotosalen. The following quantities are for orientation purposes. The skilled person may readily find concentrations and doses to be applied which achieve the effect of rendering cells apoptotic. The concentration of riboflavin-phosphate can be 1 μM to 100 μM. The concentration of amotosalen can be 50 μM to 500 μM. The light dose accompanying the afore-mentioned riboflavin or amotosalen can be 1 J/cm² to 10 J/cm². The corresponding light can be UVA or blue light. The concentration of 8-MOP can be 0.2 μM to 2.5 M (or 43 ng/ml to 540 ng/ml). The accompanying light dose can be 0.5 J/cm² to 5 J/cm². The light can be UVA or blue light.

The methods of the invention, or specific steps of the methods, can be practiced in a bag such as a plastic bag. If plastic materials are considered one may use bags made of plastic films based on: polyolefin, polyethylene, fluoropolymer, polyvinyl chloride, ethylene-vinyl acetate-copolymer, ethylene vinyl alcohol, polyvinylidene fluoride, or other plastic films approved for medical use. In a preferred embodiment of the present invention, the bag is made of ethylen-vinylacetate-copolymer. The bag may be made of a material that provides a degree of transparency such that the sample or cell mixture can be irradiated with visible or UV light.

The invention is now described with respect to some specific examples which, however, are for illustrative purposes and not to be construed in a limiting manner.

### Experiments

Experiment 1—Production of phDC

All studies were performed with blood donated by healthy human volunteers. Peripheral blood was collected into 1:100 5,000 U/mL heparin (Mckesson Packaging Services), and platelet-containing PBMC isolated by density gradient centrifugation over Isolymph (CTL Scientific Supply Corp.) following the manufacturer's protocol. Autologous plasma (also containing platelets) was collected and reserved. Washed PBMC and platelets were resuspended in autologous plasma, and incubated for 1 hr either in the Transimmunization (TI) chamber or clinical ECP plate.

In the TI chamber, the cells were passed through using a syringe pump, at a rate of 0.09 mL/min. Following plate passage, cells were collected, and the TI chamber washed with 100% FBS at 0.49 mL/min while being physically perturbed by flicking or tapping the plate surface to help detach any adherent cells from the chamber. In the clinical ECP plate, cells were passed at a flow rate of 24 mL/min, followed by a 100 mL/min wash with human AB serum (Lonza BioWhittaker) with physical perturbation by flicking or tapping the plate surface, to help detach any adherent cells. PBMC passed through either the TI chamber or the ECP plate were collected, washed, and cultured overnight under standard conditions in RPMI without phenol-red (Gibco, Carlsbad, CA) supplemented with 15% Human AB serum (Lonza BioWhittaker), 1% penicillin/streptomycin (Invitrogen, Carlsbad, CA), and 1% L-glutamine (Invitrogen, Carlsbad, CA). The following day, physiological dendritic cells were harvested (including harvest of any attached cells by scraping).

### Experiment 2: ECP Leukemia Haploidentical Transplant Trial-Schematic Design and Expected Result

1. The mother (future donor) is tolerized to paternal antigens through infusion of father's ECP-treated blood
2. A check is performed that the mother is indeed tolerized to paternal antigens (reduced MLR reaction to father's cells, no antibodies against father's HLA type)
3. Transplant bone marrow from treated mother to leukemic child
4. Evaluate bone marrow engraftment in child
5. Evaluate trial endpoints-bone marrow engraftment, GvHD incidence and severity, cancer relapse incidence

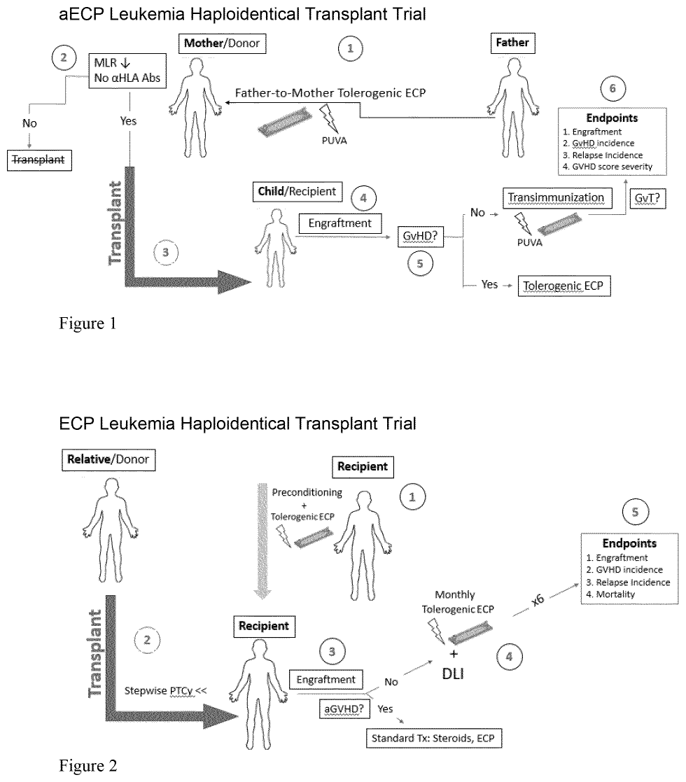

The above described treatment is depicted in FIG. 1.

Applying the above treatment, the child is not developing GvHD or shows reduced symptoms of GvHD as the transplant was pre-tolerized.

### Experiment 3: ECP Leukemia Haploidentical Transplant Trial-Schematic Design and Expected Result

1. Before haplotype bone marrow transplant or any immune-ablative pre-treatment, the recipient is treated with Tolerogenic ECP (following Francine Foss's trial design for fully matched transplants, see Experiment 1)
2. The transplant is conducted, but there is a reduction of post-transplant cyclophosphamide dose (PTCy).
3. Evaluate bone marrow engraftment
4. Evaluate trial endpoints-bone marrow engraftment, GvHD incidence and severity, cancer relapse incidence

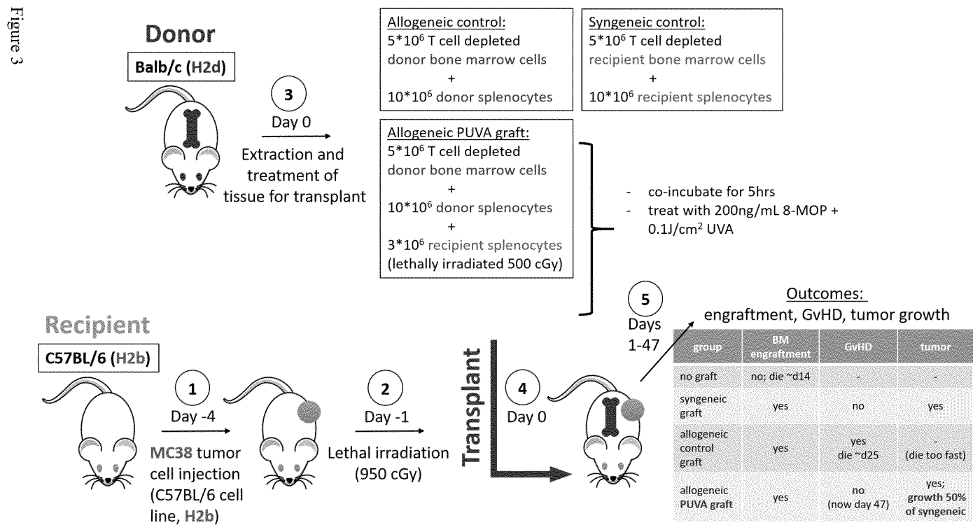

The above described treatment is depicted in FIG. 2.

Applying the above treatment, a reduction in PTCy is possible in order to preserve anti-tumor immunity. PTCy may be entirely omitted and replaced with ECP.

### Experiment 4: Prevention of GvHD by PUVA Treated Dendritic Cells

### Materials and Methods

1 On day −4, the future graft recipient mice (C57BL/6, H2b MHC haplotype) is inoculated subcutaneously (flank) with a C57BL/6 tumor (MC38 colon carcinoma).
2 On day −1, the future graft recipient (tumor-bearing) receives a lethal dose (950 cGy) of gamma irradiation that ablates his own immune system.
3 On the day of transplant (day 0), tissues are prepared for transplant.
   a. In a no-graft control, the irradiated C57BL/6 mouse does not receive any transplant.
   b. In a syngeneic control, the native C57BL/6 immune system is reconstituted by giving back C57BL/6 bone marrow and splenocytes
   c. In an allogeneic control graft, the recipient is reconstituted with fully mismatched bone marrow and splenocytes from a Balb/c donor mouse (H2d MHC haplotype).
   d. In an allogeneic PUVA graft, Balb/c bone marrow and splenocytes (containing dendritic cells) are incubated for 5 hrs with lethally irradiated C57BL/6 splenocytes, and then treated in a Petri dish with a very low dose of PUVA (200 ng/mL 8-MOP, 0.1 J/cm² UVA).
4 The prepared tissues are transplanted into the irradiated tumor-bearing recipient.
5 The recipient is then monitored for bone marrow engraftment (survival, later confirmed by blood analysis), GvHD, and tumor growth.
   The mice that are irradiated but do not receive any graft uniformly die by day 14.
   The syngeneic control mice engraft well, do not develop GvHD (expected, as the graft is a full match), and grow large tumors.
   The allogeneic control mice engraft well, develop severe GvHD that is uniformly lethal ˜day 25 (expected, as graft is a full mismatch), and tumor growth is difficult to assess since it is relatively slow, and lethality is fast.
   The allogeneic PUVA mice engraft well, do not show any signs of GvHD (day 47), and although tumors grow, the growth rate appears to be at ˜50% of syngeneic control mice, i.e. it is partially controlled by GvT effect.

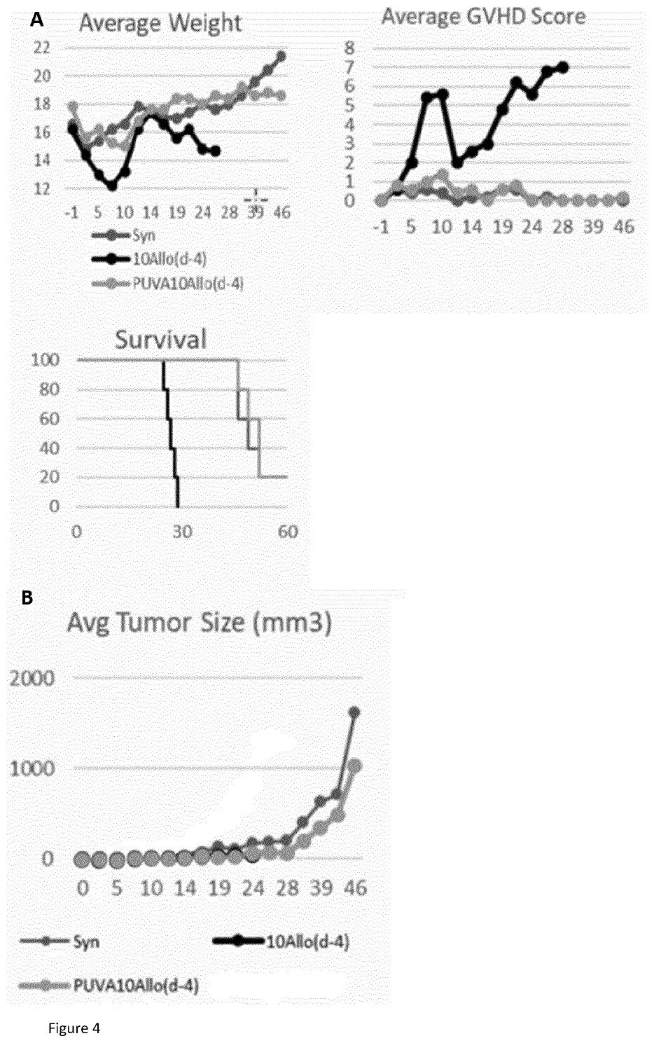

The results are depicted in Table 1 below as well as FIGS. 3 and 4. FIG. 3 also schematically depicts the above described method.

### Results

TABLE 1
Results of different treatment groups

| | Bone marrow | | |
|---|---|---|---|
| Group | engraftment | GvHD | Tumor |
| no graft | no; die ~d 14 | — | — |
| syngeneic | yes | no | yes |
| graft | | | |
| allogeneic | yes | yes | — |
| control graft | | die ~d 25 | (die too fast) |
| allogeneic | yes | no | yes; |
| PUVA graft | | (day 47) | growth 50% |
| | | | of syngeneic |

The inventors surprisingly found that a transplant treated with a low dose of 8-MOP/UVA, i.e. according to the method of the invention, prevents GvHD. The rationale is that healthy dendritic cells which took up dying dendritic cells provide tolerance to the graft in the recipient.

Experiment 5: Effect of Pre-Transplant Infusion of Donor Splenocytes Treated with Extracorporeal Photopheresis (ECP) on Cardiac Transplant Survival

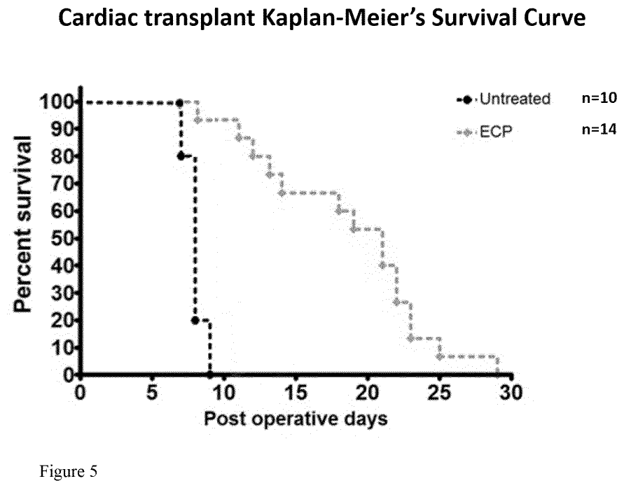

Donor mice were BALB/c (H-2^{d}), male, 8-14 weeks. Recipient mice were C57BL/c, male, 10-14 weeks (n=24). Donor mice splenocytes were collected and split into two groups. Donor cells of group 1 were subjected to shear force in a flow chamber (“untreated” in FIG. 5). Donor cells from group 2 were subjected to shear force in a flow chamber and ECP (“ECP” in FIG. 5). 10 recipient mice received untreated donor cells, while 14 recipient mice received ECP treated donor cells.

Treatment of the donor cells prior to infusion in the recipient mice was as follows:

A flow chamber was coated with platelet-rich-plasma (PRP) by introducing a 0.4 mL PRP fraction into the flow chamber for 60 min at 37° C.

Donor splenocytes were injected into the flow chamber using a 60 ml syringe at a concentration of 400 million cells in 13.33 mL.

ECP was conducted by adding 8-MOP to the flow chamber at an amount of 200 μL in 20 mL PBS per 100 million cells (200 ng/mL). The cells were exposed to UVA for 200 s with ˜20-22 mW/cm². Recipient mice received the treated donor cells at approximately 50 million cells per recipient mouse.

A cardiac transplant was conducted 7 days after infusion of the recipient mice with the donor cells. Outcome is reported in FIG. 5 as percent survival after surgery in days. As can be seen from FIG. 5, recipient mice which received untreated donor cells (i.e. group 1, donor cells only subjected to shear force) dies at around day 9 after surgery while recipient mice which received ECP donor cells (i.e. group 2, donor cells subjected to shear force and ECP) lived up to 29 days after surgery. Thus, an allogeneic transplant is tolerated significantly better in a recipient if the recipient received ECP treated donor cells, such as ECP treated splenocytes prior to transplantation (splenocytes include healthy dendritic cells). It can be concluded that the pre-treatment of a recipient with ECP treated donor cells leads to long term allograft survival and donor specific tolerance.

### Experiment 6: Extracorporeal Photopheresis for the Prevention of Acute GVHD in Patients Undergoing Standard Myeloablative Conditioning and Allogeneic Hematopoietic Stem Cell Transplantation

### Materials and Methods

### Subjects

The study and consent forms were approved by the institutional review boards, or equivalent body, of all participating sites. All subjects signed approved consent forms before beginning study treatment. Eligibility criteria included subjects 18-60 years of age with hematologic malignancies and organ function that did not preclude treatment with a myeloablative preparative regimen and allogeneic HCT. Subjects were eligible for the study if they had a diagnosis of a hematologic malignancy for which a treatment option would be an allogeneic bone marrow or PBSC transplantation. Subjects could be enrolled whether their disease was in remission or not, or after a first or second relapse of disease. Subjects were required to weigh at least 40 kg, have a platelet count above 20 000/cmm and no known sensitivity to psoralen or citrate products. Subjects had to have a related donor that was serologically or molecularly matched at all HLA-A, B and DR loci or mismatched at one HLA-A or -B locus but molecularly matched at HLA-DR loci, or have an unrelated donor that was molecularly matched at HLA-A, -B, and DR loci. As enrollment occurred from October of 2002 through January of 2004, HLA-C matching was not routinely performed. Preparative regimen and prophylaxis against GVHD Subjects received CY (60 mg/kg per day for 2 consecutive days) and TBI (10-13.5 Gy delivered over 3 or 4 days in fractionated doses.). GVHD prophylaxis was CSP 3-5 mg/kg IV beginning D-1 and adjusted to keep trough levels between 200 and 600 ng/mL. The CSP was changed to oral administration when clinically tolerated and tapered no sooner than D 100, except in subjects who had relapse or intolerance to CSP. Subjects who received their HCT from a matched related donor received MTX 10 mg/m² IV on day 1, and 5 mg/m2 on days, 3, 6, and 11, whereas subjects who had a mismatched related donor or a matched unrelated donor received MTX 15 mg/m² on day 1, and 10 mg/m² on days 3, 6, and 11. The dosing of MTX was based on agreement among the investigators to provide uniform prophylaxis for acute GVHD. Supportive care and prophylactic anti-microbial treatment were given per each study site's institutional guidelines.

### Extracorporeal Photopheresis

ECP was performed using the UVAR XTS machine (Therakos, Exton, PA, USA) as described earlier (Miller et al., 2004). In general, collection of at least 1500 mL of buffy coat blood was performed for each treatment before using methoxsalen solution (UVADEX Therakos) which was injected into the recirculation bag of the ECP circuit after collection of the buffy coat was complete, but before the photoactivation process. After completion of photoactivation, the treated buffy coat was reinfused into the subject. Patients received ECP on 2 consecutive days within 4 days before beginning the preparative regimen.

### GVHD and Adverse Event Grading

Grading of adverse events were performed according to established criteria (Common Terminology Criteria for Adverse Events. Version 3.0. Dec. 12, 2003). The modified Seattle-Glucksberg criteria were used for staging of acute GVHD (Glucksberg et al., 1974), and the diagnostic criteria of limited and extensive chronic GVHD were as described by Schulman et al., 1980. To ensure uniformity in the diagnosis of acute and chronic GVHD, investigators underwent training on the diagnostic criteria before the initiation of the trial. Each investigator employed appropriate diagnostic methods at his/her study site to determine the presence of acute or chronic GVHD.

### Statistics

Study analysis. The primary analysis was the incidence rate of grades II-IV acute GVHD in the first 100 days after transplantation and was calculated using the cumulative incidence function to accommodate the competing risk of death without the development of acute GVHD. Similarly, the cumulative incidence method was used to compute the incidence rates for chronic GVHD, transplant-related mortality (TRM), and relapse to accommodate competing risks. The probabilities of overall survival (OS) and disease-free survival (DFS) were described using Kaplan-Meier product limit estimates with 95% confidence intervals. Treatment failure (death or relapse) was the event used in the DFS assessment. Descriptive statistics, such as median time to event, were also calculated.

Comparison with historical controls. Historical controls were identified using the database maintained by the CIBMTR. The CIBMTR is a research affiliation of the International Bone Marrow Transplant Registry (IBMTR) at the Medical College of Wisconsin, and the National Marrow Donor Program that comprises a voluntary working group of more than 450 transplantation centers worldwide that contribute detailed data on consecutive allogeneic and autologous HCTs to a coordinating statistical center. Participating centers are required to report all transplants consecutively and compliance is monitored by on-site audits. All patients in the database are followed longitudinally, and yearly evaluations are included. Computerized checks for errors, physicians' review of submitted data, and on-site audits of participating centers ensure data quality. Observational studies conducted by the CIBMTR during this time period were done with a waiver of informed consent and in compliance with HIPAA regulations as determined by the Institutional Review Board and the Privacy Officer of the Medical College of Wisconsin. The CIBMTR collects data at two levels: registration and research. Registration data include disease type, age, sex, pre-transplant disease stage and chemotherapy responsiveness, date of diagnosis, graft type (bone marrow- and/or blood-derived stem cells), high-dose conditioning regimen, post transplant disease progression and survival, development of a new malignancy and cause of death. Requests for data on progression or death for registered patients are at 6-month intervals. All participating CIBMTR sites contribute registration data. Research data are collected on subset of registered patients selected using a weighted randomization scheme to ensure representativeness and include detailed disease, pre-transplant and post transplant clinical information. Historical controls for this study were derived from the Research database. ECP study subjects were transplanted between 2002 and 2004. CIBMTR controls were selected by applying the eligibility criteria for this study to subjects transplanted between 1997 and 2004. The longer time frame for the controls was used to ensure adequate numbers of subjects for adjusted comparisons with reasonable statistical power. Outcome data for both study and control subjects were censored at 1 year to adjust for the differences in length of follow-up. As study subjects and controls were transplanted in two different time periods, the year of transplant (1997-1999 versus 2000-2004) was examined for potential impact on outcomes of controls. There was no difference in 1-year outcomes of control subjects transplanted in 1997-1999 versus those transplanted in 2000-2004. Study subjects and controls were compared using multi-variate Cox regression analyses. Tests for proportionality were performed by adding a time-dependent covariate. These tests indicated that the proportionality assumptions were valid. A stepwise backward method was used to identify significant covariates (other than use of ECP) associated with outcomes. The variables considered in model building were age, gender, race, donor relationship, HLA-matching, graft type, disease type, disease status at transplant, and CMV serologic status. Treatment effect (ECP) was included in every step of model building. Tests for potential interactions between ECP treatment and other significant covariates revealed no significant interactions. One-year adjusted probabilities of overall and DFS were estimated from the final Cox models, stratified on treat-ment received and weighted by the pooled sample proportion value for each prognostic factor. These adjusted probabilities estimate likelihood of outcomes in populations with similar prognostic factors.

### Results

### Subject and Donor Characteristics

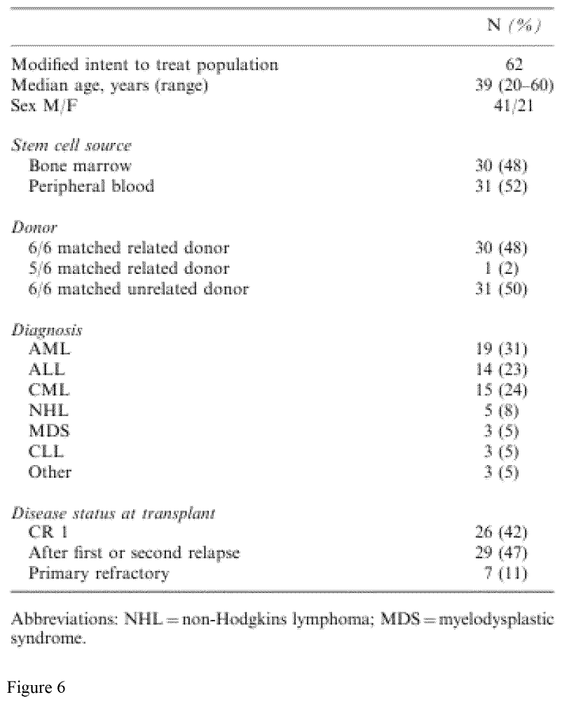

Sixty-six subjects were enrolled in the ECP study. Nine study centers enrolled from 1 to 16 subjects each (mean of seven subjects per study center). However, 3 subjects did not receive study treatment with ECP; one patient with-drew study consent, one patient had a delay in transplant, and one center had mechanical problems with the ECP instrument. One subject received ECP but was not transplanted because of rapid progression of disease. After the completion of the study 62 subjects were considered to be evaluable in the modified intent-to-treat population dataset. Two of these subjects received only one ECP treatment before starting the preparative regimen because of subsequent mechanical problem with the ECP instrument. Subject, disease, donor characteristics, and disease status information are summarized in FIG. 6.

### Engraftment

One subject did not engraft and died of respiratory failure (cause unknown) at Day 28. All other subjects had satisfactory neutrophil and platelet recovery and there were no late graft failures. The median (range) times to achieve a neutrophil count 4500/mL were 21 (14-39) days for subjects receiving bone marrow grafts and 14 (13-28) days for those receiving peripheral blood grafts. Corresponding times to achieve platelets 420 000/cmm were 14 (11-43) and 14 (6-56) days, respectively.

### GVHD

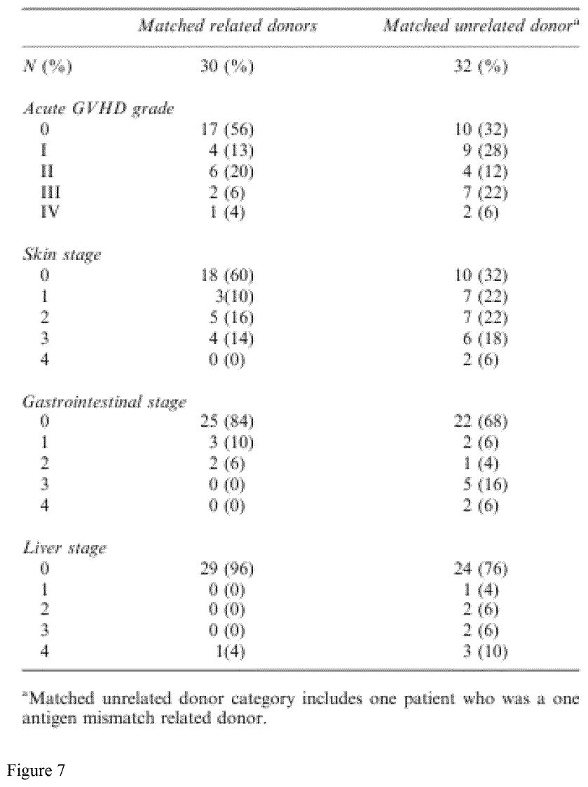

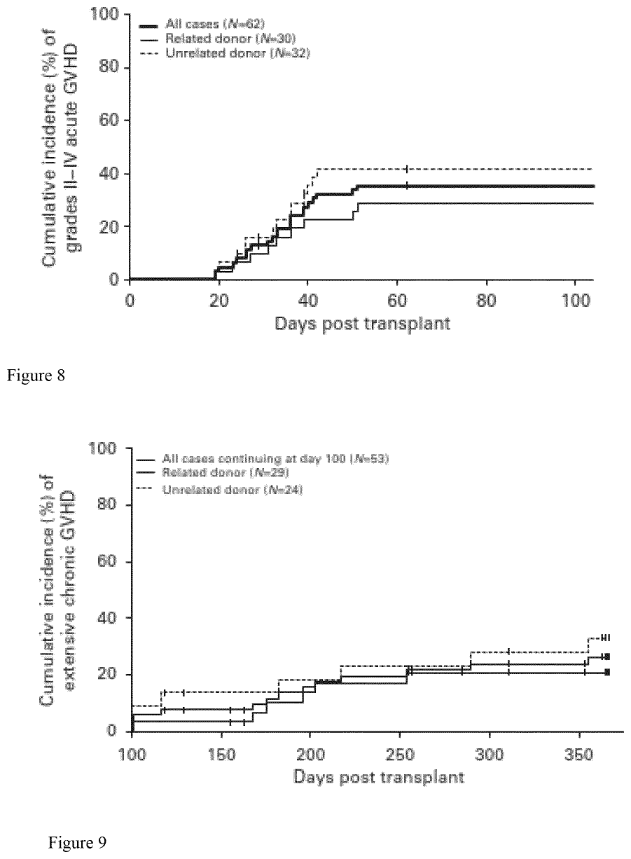

Grades II-IV acute GVHD developed in 22 (36%) of the 62 subjects, including 9 (30%) of 30 related donor HCT recipients and 13 (41%) of 32 matched unrelated or one HLA antigen mismatched related donor HCT recipients (FIG. 7). The skin was the organ most frequently and most severely involved with 38% of subjects having stage 2 or higher skin involvement, whereas 16% and 13% of subjects had stage 2 or higher involvement of the gastrointestinal tract or liver, respectively (FIG. 7). The 100-day cumulative incidence of grades II-IV acute GVHD was 35% (95% CI, 23-48%) (FIG. 8). Forty (66%) patients had biopsy proven acute GVHD. No patients were described as having early chronic GVHD before day 100 or late acute GVHD after day 100. The median time to first diagnosis and maximal grade of grades II-IV acute GVHD was 35 days for both, with a range of 18-52 days for first diagnosis and 22-96 days for maximal grade. Fifty-three subjects were evaluable for chronic GVHD. Seven patients died before day 100 (acute GVHD 3, idiopathic pneumonia 1, multiorgan system failure 3), and two patients had insufficient data collected to determine whether chronic GVHD occurred. Chronic GVHD occurred in 21 (40%) of 53 subjects (limited: 8 (15%); extensive: 13 (25%)). The 1-year cumulative incidence of limited and extensive chronic GVHD was 38% (95% CI, 21-47%) (FIG. 9).

### Toxicity

The most frequent serious adverse events that occurred during the study were fever 8 (13%), febrile neutropenia 4 (7%), and multiple organ system failure 3 (5%). Adverse events directly attributable to the ECP included two subjects who experienced hypotension while undergoing ECP. CMV reactivation occurred in 17 (27%) subjects, with two subjects having CMV disease in lung tissue documented in biopsy specimens from bronchoscopy. Two (3%) subjects had systemic fungal infections while participating in the study, one occurring 19 days post transplant and the other 9 months post transplant.

### Survival

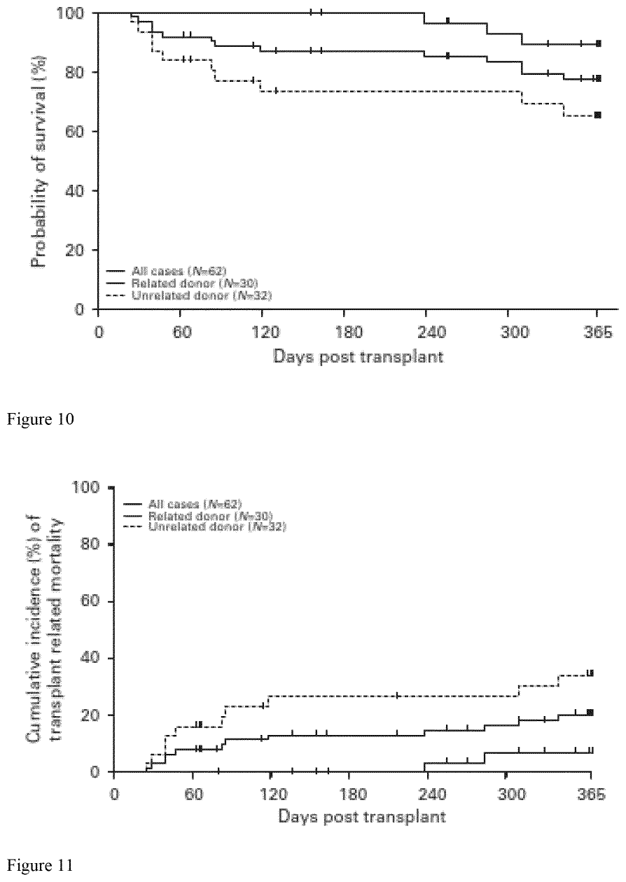

Median follow-up of surviving patients is 371 days (range, 366-643), with 48 (77%) of 62 subjects surviving. Kaplan Meier estimates of survival at day 100 and 1-year post transplant were 89% (95% CI, 78-97%) and 77% (95% CI, 64-86%), respectively. One-year probabilities of OS for related and unrelated donor HCT recipients were 89% (95% CI, 70-96%) and 66% (95% CI, 46-80%), respectively (FIG. 10). One-year probabilities of DFS were 69% (95% CI, 64-86%) for all patients, 79% (95% CI, 59-90%) after related donor HCT and 60% (95% CI, 40-75%) after unrelated donor HCT. Relapse occurred in 7 (11%) patients. Fourteen (23%) subjects died: 3 after related and 11 after unrelated donor transplants. Causes of death were relapse (1), acute GVHD (3), chronic GVHD (1), infection (4), multiple organ system failure (3), and idiopathic pneumonitis (2). The 1-year cumulative incidence of TRM was 21% (95% CI, 11-31%); the cumulative incidences after related and unrelated donor transplants were 10% (95% CI, 1-20%) and 31% (95% CI, 18-50%), respectively, FIG. 11).

ECP Study Subjects Compared with CIBMTR Controls

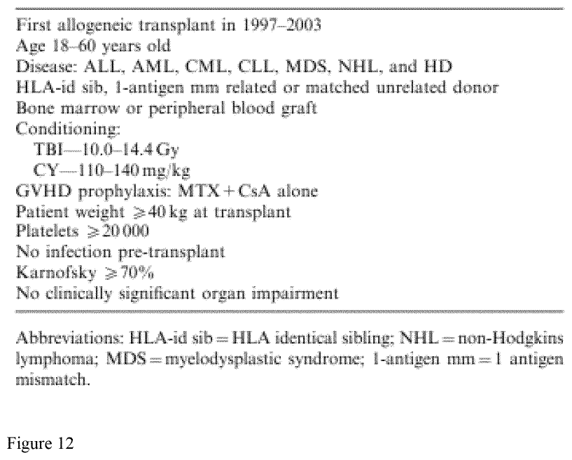

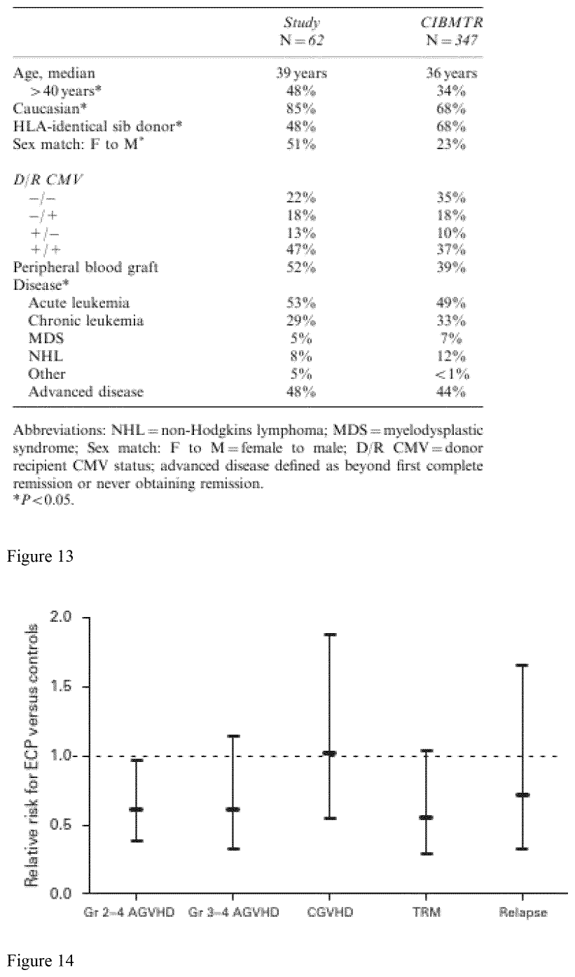

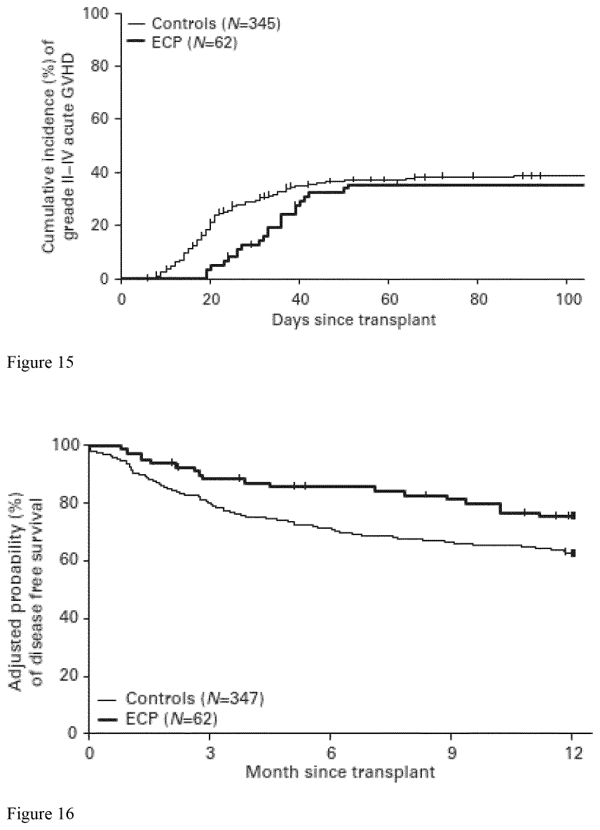

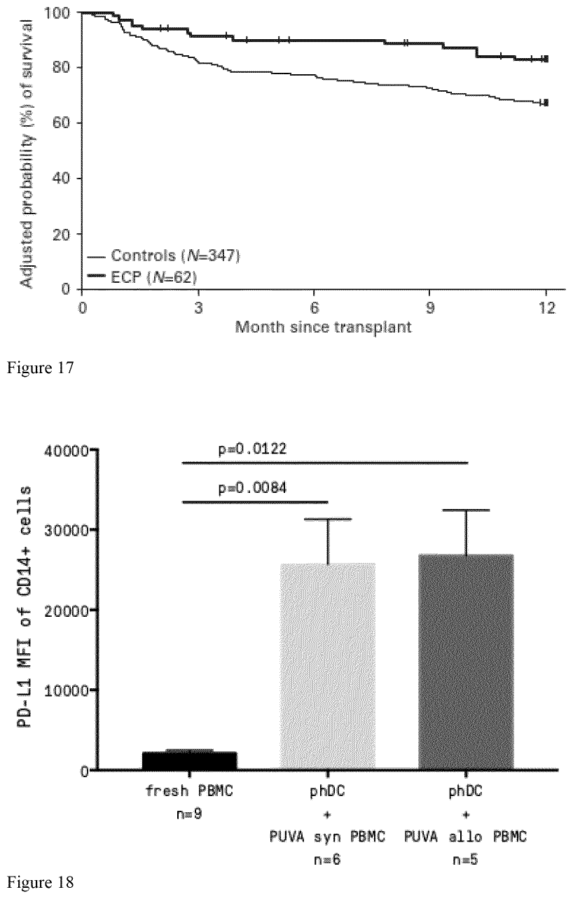

The CIBMTR database was used to search for historical controls with characteristics similar to the study subjects. Control subjects were required to meet the eligibility criteria defined in FIG. 12. A total of 347 control subjects were identified. Their characteristics are compared with those of the study subjects in FIG. 13. ECP-treated subjects were more likely than controls to be older than 40 years, to be Caucasian, and to have an unrelated donor. The distribution of underlying diseases was also significantly different. The potential impact of these differences was considered in multivariate analyses. Multivariate analysis revealed a significantly lower rate of developing grades II-IV acute GVHD (relative risk [RR], 0.61; 95% CI, 0.38-0.97) (P=0.04) in ECP-treated subjects compared with historical controls (FIG. 14). This lower rate was due to a substantial delay in the onset of acute GVHD in the ECP-treated subjects rather than an absolute decrease in the incidence (FIG. 15). The cumulative incidence probability of acute GVHD grades II-IV was 36% (95% CI, 25-48%) in the study patients and 39% (95% CI, 33-44%) in the historical control cohort (FIG. 15). There was also less TRM in the ECP-treated subjects compared with the historical controls, but this was not statistically significant (RR, 0.55; 95% CI, 0.29-1.04) (P=0.065). There was no difference in veno-occlusive disease or interstitial pneumonitis between the groups. However, opportunistic infections occurred in 85 (24%) of the control patients and 5 (8%) of the study patients (P=0.008). The adjusted probabilities of DFS and OS were significantly higher in the ECP-treated subjects than in the historical controls (FIGS. 16 and 17). The adjusted DFS rates at 1 year were 74% (95% CI, 62-82%) in the ECP-treated subjects and 63% (95% CI, 58%-67%) in the historical control cohort (RR of treatment failure [relapse or death], 0.60; 95% CI, 0.36-0.99) (P=0.045). The adjusted OS rates at 1 year were 83% (95% CI, 72-90%) in the ECP-treated subjects and 67% (95% CI, 62-71%) in the historical controls (RR of mortality, 0.44; 95% CI, 0.24-0.80) (P=0.007).

### DISCUSSION

Collaboration with the CIBMTR was undertaken at the completion of the study to identify suitable historical controls for comparison with ECP study subjects to put the results of this single Phase II study in perspective. Controls were selected using the eligibility criteria for the patients in this study. However, there were some differences in the distribution of characteristics between the groups as described in FIG. 13, with ECP study subjects more likely to be older and to receive an unrelated donor graft, both associated with increased risk of GVHD. Despite the presence of these key demographic differences favoring the historical control group, acute GVHD occurred more slowly in the ECP-treated cohort. Multivariate analysis, adjusting for the differences in prognostic factors, revealed a significant difference in rate of acute GVHD (grades II-IV) between the groups (FIG. 15), with a significant delay in the time to onset of acute GVHD. Although the absolute incidence of acute GVHD was not lower with ECP, multivariate analysis revealed a trend to less TRM, less treatment failure (relapse and TRM), and higher overall and DFS in the ECP study cohort compared with the historical control cohort. Regimen-related toxicities were similar between the groups, except for significantly less opportunistic infections in the ECP-treated patients compared with historical controls. Later onset of acute GVHD may itself be beneficial, as it may permit more recovery from the preparative regimen and transplant procedure, and a higher degree of immune reconstitution, allowing these patients to better tolerate the side effects of corticosteroids, have less end organ damage, and to overcome infections. ECP did not appear to suppress the allo-immune-mediated graft-versus-malignancy effect as relapse rates were not significantly different between the two groups, though the follow-up time for the evaluation of relapse was short.

Experiment 7: Amelioration of Autoimmune Diseases Using Tolerogenic phDC

In this example, an animal model is used to evaluate amelioration of an autoimmune disease using tolerogenic phDC.

A number of accepted animal models for autoimmune disease are available; exemplary animal models are shown in Table A above. If necessary, the autoimmune disease is induced prior to treatment with tolerogenic phDC or controls. Animals are examined and scored according to relevant clinical criteria for the animal model. In some examples, the animals are divided into four treatment groups, as follows:
a) untreated animals
b) treated with healthy phDC presenting autoantigens
c) treated with 8-MOP/UVA-damaged apoptotic phDC presenting autoantigens
d) treated with healthy phDC that have internalized 8-MOP/UVA-damaged apoptotic phDC containing autoantigens

The phDC for the above treatment groups can be generated as follows:
b) Healthy phDC presenting autoantigens:
Monocytes are obtained from healthy syngeneic animals, passed through the TI plate as described (Ventura et al. J Vis Exp. 2019 May 17; 147), and incubated with autoantigen overnight to ensure antigen uptake.
c) 8-MOP/UVA-damaged apoptotic phDC presenting autoantigens: Monocytes are obtained from healthy syngeneic animals and passed through the TI plate as above, then exposed to 8-MOP and UVA at a dose sufficient to induce cell damage, and incubated with autoantigen overnight.
d) Healthy phDC that have internalized 8-MOP/UVA-damaged apoptotic phDC containing autoantigens:
Cells from group c) are combined with an equal number of fresh monocytes, passed through the TI plate as above, and co-incubated overnight to ensure uptake of 8-MOP/UVA-damaged phDC containing autoantigens by healthy phDC.

The phDC generated as described above are administered to the mice in the appropriate groups intravenously, typically at least three times, in appropriate doses (e.g., of at least 1×10⁶ cells/animal), under appropriate time intervals for the animal model.

Animals are monitored (e.g., daily) for the autoimmune disease for a suitable period of time for the particular model. Additionally, animals may be sacrificed at different time points during the experiment:
(i) before disease induction, i.e., healthy animals;
(ii) following disease induction, but without treatment, i.e., immunized animals;
(iii) between the second and third vaccination, i.e., an intermediate time point;
(iv) at the end of the experiment, i.e., end point

Following euthanasia, samples from the animals (e.g., spleens and inguinal and axillary lymph nodes) are obtained, dissociated into a single-cell suspension, and used to assess the immune response to immunization and treatment. Standard immune response assays include immune cell phenotype analysis by cell surface or intracellular flow cytometry, inflammatory or anti-inflammatory cytokine secretion assays (e.g., ELISA, Luminex, ELISpot), and T cell proliferation in response to autoantigen re-stimulation (e.g., carboxyfluorescein succinimidyl ester (CFSE) dilution).

Exemplary results will include:
a) untreated animals:
   Progressive disease consistent with what is expected in the model.
b) treated with healthy phDC presenting autoantigens:
   Progressive disease, possibly more severe than is expected in the model, due to additional immunizing effects of healthy phDC.
c) treated with 8-MOP/UVA-damaged apoptotic phDC presenting autoantigens:
   Progressive disease, possibly less severe than is expected in the model, due to some tolerogenic effect of 8-MOP/UVA-damaged apoptotic phDC being taken up by healthy DC in the injected animal.
d) treated with healthy phDC that have internalized 8-MOP/UVA-damaged apoptotic phDC containing autoantigens:
   Significantly reduced disease, due to tolerogenic effect of healthy phDC that have taken up 8-MOP/UVA-damaged apoptotic phDC carrying autoantigens, and are thus able to control and reduce disease.
      Experiment 8: Amelioration of Autoimmune Diseases Including Multiple Sclerosis (MS) Using Tolerogenic phDC

In this example, a mouse model is used to evaluate whether tolerogenic phDC ameliorate an autoimmune disease such as MS.

Experimental autoimmune encephalomyelitis (EAE) is a widely accepted mouse model of human MS, with many similarities to human clinical disease. The murine EAE model is characterized by progressive paralysis, CNS inflammation, and demyelination, and is mainly mediated by myelin-specific CD4+ T cells, although CD8+ cells and B cells also play a role. EAE mice can thus be used to model tolerance induction by dendritic cells in an autoimmune disease setting.
1. EAE is induced in 11-13-week-old female C57BL/6 mice by immunization with myelin oligodendrocyte glycoprotein (MOG) peptides MOG35-55 or MOG1-125 in Complete Freund's adjuvant (CFA) emulsion, followed by administration of pertussis toxin (PTX) in PBS, following standard protocols known in the art.
2. EAE is expected to develop 8 to 18 days after immunization. After disease induction, all animals are examined daily for well-being and scored according to the following standard EAE clinical criteria: 0, asymptomatic; 0.5, loss of tone in the distal half of the tail; 1, loss of tone in the entire tail; 1.5, hind limb weakness; 2, hind limb paralysis; 2.5, hind limb paraplegia; 3, forelimb weakness; 4, quadriparesis; 4.5, severe quadriparesis; 5, quadriplegia; and 6, death. Treatment is initiated on the first day the mean clinical score exceeds 1.0, reflecting clinically relevant disease onset in the majority of mice.
3. Mice are divided into 4 treatment groups of at least 10 animals each, as follows:
   a) untreated mice
   b) treated with healthy phDC presenting MOG antigens
   c) treated with 8-MOP/UVA-damaged apoptotic phDC presenting MOG antigens
   d) treated with healthy phDC that have internalized 8-MOP/UVA-damaged apoptotic phDC containing MOG antigens
4. The phDC for the above treatment groups will be generated as follows:
   b) Healthy phDC presenting MOG antigens:
   Monocytes are obtained from healthy syngeneic mice, passed through the TI plate as described (Ventura et al. J Vis Exp. 2019 May 17; 147), and incubated with MOG antigen overnight to ensure antigen uptake.
   c) 8-MOP/UVA-damaged apoptotic phDC presenting MOG antigens: Monocytes are obtained from healthy syngeneic mice and passed through the TI plate as above, then exposed to 8-MOP and UVA at a dose sufficient to induce cell damage, and incubated with MOG antigen overnight.
   d) Healthy phDC that have internalized 8-MOP/UVA-damaged apoptotic phDC containing MOG antigens:
   Cells from group c) are combined with an equal number of fresh monocytes, passed through the TI plate as above, and co-incubated overnight to ensure uptake of 8-MOP/UVA-damaged phDC containing MOG antigens by healthy phDC.
5. The phDC generated as described above (see 4.) are administered to the mice in the appropriate groups (see 3.) intravenously at least three times, in doses of at least 1×10⁶ cells/mouse, for instance as follows:
   on the first day the mean clinical score exceeds 1.0, reflecting clinically relevant disease onset in the majority of mice (e.g., day 13 post immunization)
   four days after the first treatment dose (e.g., day 17 post immunization)
   four days following the second treatment dose (e.g., day 21 post immunization)
6. Mice are monitored daily for EAE clinical score, until at least 4 weeks post immunization.
7. Additionally, mice may be sacrificed at different time points during the experiment:
   (i) before EAE induction, i.e., healthy mice;
   (ii) following EAE induction, but without treatment, i.e., immunized mice;
   (iii) between the second and third vaccination, i.e., an intermediate time point;
   (iv) at the end of the experiment, i.e., end point
Following euthanasia, spleens and inguinal and axillary lymph nodes are obtained, dissociated into a single-cell suspension, and used to assess the immune response to immunization and treatment. Standard immune response assays include immune cell phenotype analysis by cell surface or intracellular flow cytometry, inflammatory or anti-inflammatory cytokine secretion assays (e.g., ELISA, Luminex, ELISpot), and T cell proliferation in response to MOG peptide re-stimulation (e.g., carboxyfluorescein succinimidyl ester (CFSE) dilution).
Spinal cords are also obtained at euthanasia, fixed in 4% paraformaldehyde (PFA), and used for additional evaluation of the disease by histology and immunohistochemistry. Typical histological analysis include inflammatory foci counts, apoptotic cell counts, and extent of demyelination (immunohistochemistry for Myelin Basic Protein).

Exemplary results will include:
a) untreated mice:
   Progressive EAE disease consistent with what is expected in this model.
   Upon immunologic examination, detection of inflammatory immune cells reactive against MOG antigens.
   Upon histologic examination, signs of axonal injury and inflammatory damage consistent with the model.
b) treated with healthy phDC presenting MOG antigens:
   Progressive EAE disease, possibly more severe than is expected in this model, due to additional immunizing effects of healthy phDC.
   Upon immunologic examination, increased detection of inflammatory immune cells reactive against MOG antigens.
   Upon histologic examination, signs of more severe axonal injury and inflammatory damage.
c) treated with 8-MOP/UVA-damaged apoptotic phDC presenting MOG antigens:
   Progressive EAE disease, possibly less severe than is expected in this model, due to some tolerogenic effect of 8-MOP/UVA-damaged apoptotic phDC being taken up by healthy DC in the injected mouse.
   Upon immunologic examination, somewhat reduced detection of inflammatory immune cells reactive against MOG antigens.
   Upon histologic examination, signs of less severe axonal injury and inflammatory damage.
d) treated with healthy phDC that have internalized 8-MOP/UVA-damaged apoptotic phDC containing MOG antigens:
   Significantly reduced EAE disease, due to tolerogenic effect of healthy phDC that have taken up 8-MOP/UVA-damaged apoptotic phDC carrying MOG antigens, and are thus able to control and reduce EAE disease.
   Upon immunologic examination, detection of tolerogenic immune cells, such as Treg's, and significant reduction of inflammatory immune cells reactive against MOG antigens.
   Upon histologic examination, significantly reduced axonal injury and inflammatory damage.
      Experiment 9: Amelioration of Other Autoimmune Diseases Using Tolerogenic phDC

In this example, a suitable animal model (e.g., an animal model described in Table A above) is used to evaluate whether tolerogenic phDC ameliorate an autoimmune disease.

For example, nonobese diabetic (NOD) mice are an art-accepted model for insulin-dependent diabetes mellitus (IDDM). In this example, NOD mice are evaluated using a similar approach as is described in Experiments 7 and 8.

Animals are examined and scored according to relevant clinical criteria for NOD mice. In some examples, the animals are divided into four treatment groups, as follows:
a) untreated animals
b) treated with healthy phDC presenting autoantigens (e.g., pancreatic β-cell antigen)
c) treated with 8-MOP/UVA-damaged apoptotic phDC presenting autoantigens (e.g., pancreatic β-cell antigen)
d) treated with healthy phDC that have internalized 8-MOP/UVA-damaged apoptotic phDC containing autoantigens (e.g., pancreatic β-cell antigen)

The phDC for the above treatment groups can be generated as follows:
b) Healthy phDC presenting autoantigens (e.g., pancreatic β-cell antigen):
Monocytes are obtained from healthy syngeneic animals, passed through the TI plate as described (Ventura et al. J Vis Exp. 2019 May 17; 147), and incubated with autoantigen overnight to ensure antigen uptake.
c) 8-MOP/UVA-damaged apoptotic phDC presenting autoantigens (e.g., pancreatic β-cell antigen):
Monocytes are obtained from healthy syngeneic animals and passed through the TI plate as above, then exposed to 8-MOP and UVA at a dose sufficient to induce cell damage, and incubated with autoantigen (e.g., pancreatic β-cell antigen) overnight.
d) Healthy phDC that have internalized 8-MOP/UVA-damaged apoptotic phDC containing autoantigens (e.g., pancreatic β-cell antigen):
Cells from group c) are combined with an equal number of fresh monocytes, passed through the TI plate as above, and co-incubated overnight to ensure uptake of 8-MOP/UVA-damaged phDC containing autoantigens (e.g., pancreatic β-cell antigen) by healthy phDC.

The phDC generated as described above are administered to the mice in the appropriate groups intravenously, typically at least three times, in appropriate doses (e.g., of at least 1×10⁶ cells/animal), under appropriate time intervals for the animal model.

Animals are monitored for diabetic phenotypes. Additionally, animals may be sacrificed at different time points during the experiment:
(i) before disease induction, i.e., healthy animals;
(ii) following disease induction, but without treatment, i.e., immunized animals;
(iii) between the second and third vaccination, i.e., an intermediate time point;
(iv) at the end of the experiment, i.e., end point

Following euthanasia, samples from the animals (e.g., spleens and inguinal and axillary lymph nodes) are obtained, dissociated into a single-cell suspension, and used to assess the immune response to immunization and treatment. Standard immune response assays include immune cell phenotype analysis by cell surface or intracellular flow cytometry, inflammatory or anti-inflammatory cytokine secretion assays (e.g., ELISA, Luminex, ELISpot), and T cell proliferation in response to autoantigen re-stimulation (e.g., carboxyfluorescein succinimidyl ester (CFSE) dilution).

Exemplary results will include:
a) untreated animals:
   Progressive disease consistent with what is expected in the NOD model.
b) treated with healthy phDC presenting autoantigens:
   Progressive disease, possibly more severe than is expected in the NOD model, due to additional immunizing effects of healthy phDC.
c) treated with 8-MOP/UVA-damaged apoptotic phDC presenting autoantigens:
   Progressive disease, possibly less severe than is expected in the NOD model, due to some tolerogenic effect of 8-MOP/UVA-damaged apoptotic phDC being taken up by healthy DC in the injected animal.
d) treated with healthy phDC that have internalized 8-MOP/UVA-damaged apoptotic phDC containing autoantigens:
   Significantly reduced disease, due to tolerogenic effect of healthy phDC that have taken up 8-MOP/UVA-damaged apoptotic phDC carrying autoantigens, and are thus able to control and reduce disease.

Other autoimmune diseases can be evaluated using a similar approach as described above.

Experiment 10: Treatment of Autoimmune Diseases Using Tolerogenic phDC

Human patients having autoimmune diseases are treated using tolerogenic phDC produced as described herein.

For example, a sample of dendritic cells is obtained from a patient suffering from an autoimmune disease (e.g., MS). The dendritic cells are exposed to an apoptotic agent (e.g., the combination of a psoralen and UVA (PUVA), in particular 8-MOP and UVA). In some examples, an autoantigen is also added to the dendritic cells being exposed to an apoptotic agent. For example, for MS, any appropriate autoantigen described in Table A may be added, e.g., MBP and/or MOG. The cells are exposed to the apoptotic agent for a time and under conditions such that the cells undergo apoptosis.

The resulting apoptotic dendritic cells are then combined with physiologic dendritic cells produced as described herein, and may be co-incubated, e.g., for least 0.5 h, 1 h, 2 h, 3 h, 4 h, 5 h or 6 h prior to administration to the subject. Alternately, the combination can be directly administered to the subject directly without co-incubation. Treatment with the phDCs is expected to result in amelioration of the autoimmune disease.

Experiment 11: Increased PD-L1 Expression in Human phDC Incubated with PUVA Damaged PBMC

Human phDC were generated using the TI plate from the blood of healthy volunteers, and incubated overnight with an equal number of 8-MOP/UVA treated syngeneic PBMC (PUVA syn PBMC), or allogeneic PBMC (PUVA allo PBMC). PD-L1 expression (reported as mean fluorescence intensity, MFI, of live, CD14+phDC fraction) was measured by flow cytometry at 18 hrs, and found to be expressed at a significantly higher level on healthy phDC after co-incubation with either allogeneic or syngeneic PUVA-treated PBMC, than on precursor monocytes (FIG. 18). N=number of blood donors analyzed; p-value=unpaired t test with Welch's correction.

### Experiment 12: Decreased PD1 Expression of Responder T Cells in MLR Assay

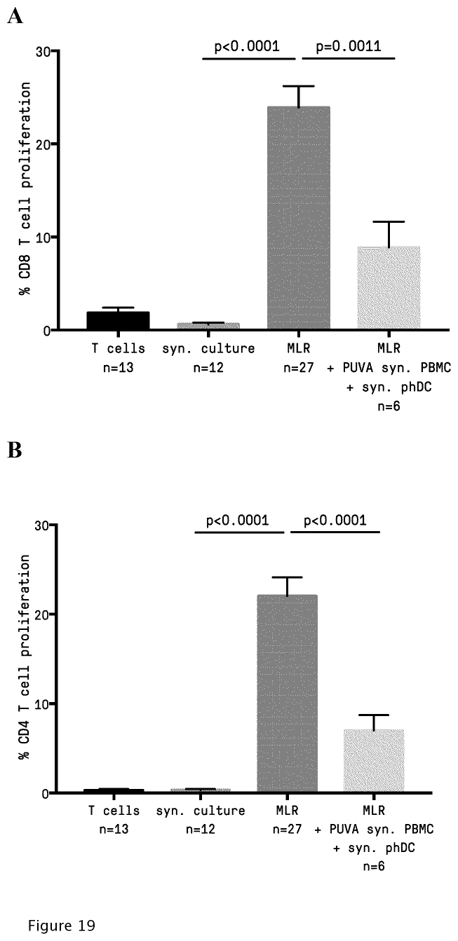

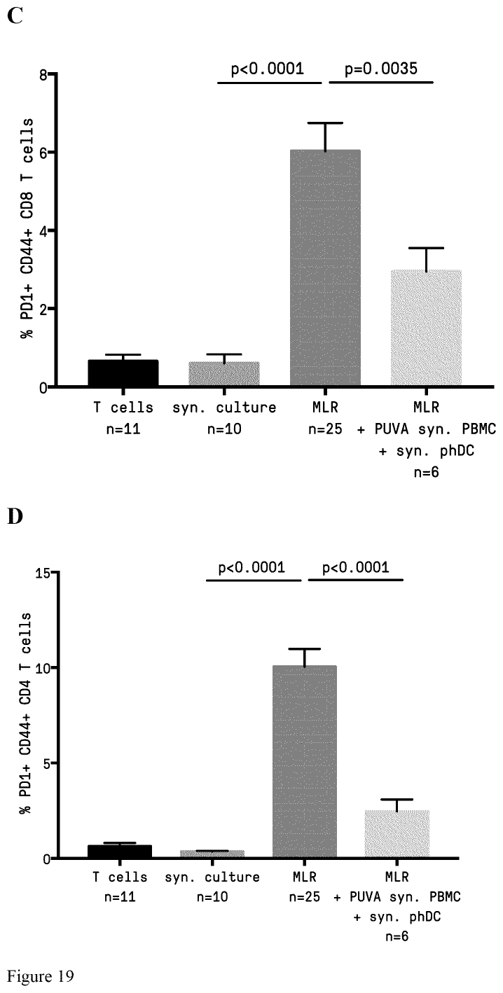

MLR (Mixed Lymphocyte Reaction) assays were set up using blood from healthy volunteer donors. Briefly, 2*10⁵ purified, CFSE-labeled responder T cells from one donor (T cells) were co-incubated with 4*10⁵ gamma-irradiated (3000 rad) stimulator PBMC either from the same donor (syn. culture) or from an unrelated donor (MLR). In order to suppress the MLR reaction, some cultures were additionally supplemented with 1*10⁵ syngeneic 8-MOP/UVA-treated PBMC, and 1*10⁵ syngeneic TI plate-passed phDC (MLR+PUVA syn. PBMC+phDC). After 5 days of culture, the proliferation of responder CD8 and CD4 T cells was assayed by measuring CFSE dilution by flow cytometry (FACS) (A, B). The activation state of responder CD8 and CD4 T cells was additionally assessed by FACS, using CD44 and PD1 expression to detect activated T cells (C, D). For both CD8 and CD4 T cells, addition of healthy phDC and PUVA-treated PBMC significantly suppressed both proliferation and activation (FIG. 19). N=number of blood donors analyzed; p-value=unpaired t test with Welch's correction.

Furthermore, the invention relates to the following embodiments.

1. A method to selectively produce tolerogenic dendritic cells, the method comprising the following steps:
a) providing dendritic cells from a donor;
b) exposing the dendritic cells of step a) to apoptotic agents;
c) providing physiologic dendritic cells from a recipient; and
d) combining the apoptotic donor dendritic cells of step b) with physiologic recipient dendritic cells from step c).

2. Method according to 1, wherein after step d), a step of co-incubating the apoptotic donor dendritic cells of step b) with the physiologic recipient dendritic cells of step c) is performed.

3. Method according to 2, wherein the step of co-incubating is performed for at least 0.5 hours (h), 1 h, 2 h, 3 h, 4 h, 5 h or 6 h.

4. Method according to 1, wherein step d) of combining the apoptotic donor dendritic cells with the physiologic dendritic cells from the recipient takes place within the recipient.

5. Method according to 1, wherein the dendritic cells of step a) are derived from an extracorporeal blood sample of the donor.

6. Method according to 1, wherein the dendritic cells of step a) have been obtained by plate passage of PBMC from the donor.

7. Method according to 1, wherein the apoptotic agents of step b) comprise psoralens and UVA, riboflavin-phosphate and UVA, and/or 5-aminolevulinic acid and light.

8. Method according to 7, wherein the psoralen is selected from the group comprising 8-MOP and amotosalen.

9. Method according to 8, wherein the psoralen is 8-MOP.

10. Method according to 1, wherein the physiologic dendritic cells of step c) have been obtained by plate passage of PBMC from the recipient.

11. Method according to 1, 2, 3, 4, 5, 6, 7, 8, 9 or 10, wherein the donor and/or recipient are mammalian, preferably human.

12. A method to selectively produce tolerogenic dendritic cells, the method comprising the following steps:
a) Providing dendritic cells from a recipient's complimentary haplo-donor;
b) Exposing the dendritic cells of step a) to apoptotic agents;
c) Providing physiologic dendritic cells from the recipient's haplo-donor; and
d) Combining the apoptotic complimentary haplo-donor dendritic cells of step b) with the physiologic haplo-donor dendritic cells of step c).

13. Method according to 12, wherein after step d), a step of co-incubating the apoptotic complimentary haplo-donor dendritic cells of step b) with the physiologic haplo-donor dendritic cells of step c) is performed.

14. Method according to 13, wherein the step of co-incubating is performed for at least 0.5 h, 1 h, 2 h, 3 h, 4 h, 5 h or 6 h.

15. Method according to 12, wherein step d) of combining the apoptotic complimentary haplo-donor dendritic cells of step b) with the physiologic haplo-donor dendritic cells of step c) takes place within the haplo-donor.

16. Method according to 12, wherein the dendritic cells of step a) are derived from an extracorporeal blood sample of the recipient's complimentary haplo-donor.

17. Method according to 12, wherein the dendritic cells of step a) have been obtained by plate passage of PBMC from the recipient's complimentary haplo-donor.

18. Method according to 12, wherein the apoptotic agents of step b) comprise psoralens and UVA, riboflavin-phosphate and UVA and/or 5-aminolevulinic acid and light.

19. Method according to 18, wherein the psoralen is selected from the group comprising 8-MOP and amotosalen.

20. Method according to 19, wherein the psoralen is 8-MOP.

21. Method according to 12, wherein the physiologic dendritic cells of step c) have been obtained by plate passage of PBMC from the recipient's complimentary haplo-donor.

22. Method according to any of 12, 13, 14, 15, 16, 17, 18, 19, 20 or 21, wherein the complimentary haplo-donor, the haplo-donor and/or the recipient are mammalian, preferably human.

23. A method to selectively produce tolerogenic dendritic cells, the method comprising the following steps:
a) Providing dendritic cells from a recipient;
b) Exposing the dendritic cells of step a) to an apoptotic agent;
c) Providing physiologic dendritic cells from the recipient; and
d) Combining the apoptotic dendritic cells of step b) with the physiologic dendritic cells of step c).

24. Method according to 23, wherein after step d), a step of co-incubating the apoptotic dendritic cells of step b) with the physiologic dendritic cells of step c) is performed.

25. Method according to 24, wherein the step of co-incubating is performed for at least 0.5 h, 1 h, 2 h, 3 h, 4 h, 5 h or 6 h.

26. Method according to 23, wherein step c) of combining the apoptotic dendritic cells of step b) with the physiologic dendritic cells of step c) takes place within the recipient.

27. Method according to 23, wherein the dendritic cells of step a) are derived from an extracorporeal blood sample of the recipient.

28. Method according to 23, wherein the dendritic cells of step a) have been obtained by plate passage of PBMC from the recipient.

29. Method according to 23, wherein the apoptotic agents of step b) comprise psoralens and UVA, riboflavin-phosphate and UVA and/or 5-aminolevulinic acid and light.

30. Method according to 29, wherein the psoralen is selected from the group comprising 8-MOP and amotosalen.

31. Method according to 30, wherein the psoralen is 8-MOP.

32. Method according to 23, wherein the physiologic dendritic cells of step c) have been obtained by plate passage of PBMC from the recipient.

33. Method according to any of 23 to 32, wherein the donor and recipient are mammalian, preferably human.

34. Method according to any of 1 to 33, wherein the transplant is an organ or stem cell transplant.

35. Tolerogenic dendritic cells obtained by a method according to any of 1 to 11.

36. Tolerogenic dendritic cells obtained by a method according to any of 12 to 22.

37. Tolerogenic dendritic cells obtained by a method according to any of 23 to 34.

38. Tolerogenic dendritic cells according to 35 to 37 for use in a method of preventing or reducing graft versus host disease.

39. A method to selectively produce tolerogenic dendritic cells, the method comprising the following steps:
a) Providing a first sample of dendritic cells obtained from a subject;
b) Exposing the dendritic cells of step a) to an apoptotic agent;
c) Providing a second sample of physiologic dendritic cells obtained from the subject; and
d) Combining the apoptotic dendritic cells of step b) with the physiologic dendritic cells of step c).

40. Method according to 39, wherein after step d), a step of co-incubating the apoptotic dendritic cells of step b) with the physiologic dendritic cells of step c) is performed.

41. Method according to 40, wherein the step of co-incubating is performed for at least 0.5 h, 1 h, 2 h, 3 h, 4 h, 5 h or 6 h.

42. Method according to 39, wherein step c) of combining the apoptotic dendritic cells of step b) with the physiologic dendritic cells of step c) takes place within the subject.

43. Method according to any of 39 to 42, wherein the dendritic cells of step a) are derived from an extracorporeal blood sample of the subject.

44. Method according to 43, wherein the dendritic cells of step a) have been obtained by plate passage of PBMC from the subject.

45. Method according to any of 39 to 44, wherein the method further comprises step a1) of incubating the dendritic cells with antigenic molecules.

46. Method according to 45, wherein the antigenic molecule is an autoantigen.

47. Method according to 45 or 46, wherein the antigenic molecule is derived from a natural source, chemically synthesized or recombinantly produced.

48. Method according to 45 or 46, wherein the antigenic molecule is derived from a cell.

49. Method according to 46, wherein the autoantigen is selected from the group comprising Rh blood group antigens, platelet integrin GpIIb:IIIa, noncollagenous domain of basement membrane collagen type IV, epidermal cadherin, streptococcal cell-wall antigens, rheumatoid factor IgG complexes with or without hepatitic C antigens, pancreatic β-cell antigen, myelin basic protein, proteolipid protein, myelin oligodendrocyte glycoprotein, desmoglein 3, glutamic acid decarboxylase, acetylcholine receptor, carboxypeptidase H, chromogranin A, glutamate decarboxylase, imogen-38, insulin, insulinoma antigen-2 and 2β, islet-specific glucose-6-phosphatase catalytic subunit related protein (IGRP), proinsulin, α-enolase, aquaporin-4, β-arrestin, S100-β, citrullinated protein, collagen II, heat shock proteins, human cartilage glycoprotein 39, La antigen, nucleosomal histones and ribonucleoproteins (snRNP), phospholipid-β-2 glycoprotein I complex, poly (ADP-ribose) polymerase, Sm antigens of U-1 small ribonucleoprotein complex, pancreatic islet cell antigens, cytoplasmic linker protein-170 (CLIP-170), Sjogren's syndrome antigen A (SS-A/Ro), Sjogren's syndrome antigen B (SS-B/La), Sjogren's lupus antigen (SL) and scleroderma antigen 70 (Scl-70)).

50. Method according to any of 39 to 49, wherein the apoptotic agents of step b) comprise psoralens and UVA, riboflavin-phosphate and UVA and/or 5-aminolevulinic acid and light.

51. Method according to 50, wherein the psoralen is selected from the group comprising 8-MOP and amotosalen.

52. Method according to 50, wherein the psoralen is 8-MOP.

53. Method according to any of 39 to 52, wherein the physiologic dendritic cells of step c) have been obtained by plate passage of PBMC from the subject.

54. Method according to any of 39 to 53, wherein the subject is mammalian, preferably human.

55. Tolerogenic dendritic cells obtained by a method according to any of 39 to 54.

56. Tolerogenic dendritic cells according to 55 for use in the treatment of autoimmune diseases.

57. Tolerogenic dendritic cells for use according to 56, wherein the autoimmune disease is selected from the group comprising multiple sclerosis, rheumatoid arthritis, juvenile rheumatoid arthritis, systemic lupus erythematosus, amyotrophic lateral sclerosis, pemphigus vulgaris, psoriasis, myasthenia gravis, thyroiditis, scleroderma, Sjogren's syndrome, thrombocytopenia purpura, cryoglobulinemia, autoimmune haemolytic anemia, insulin-dependent diabetes mellitus (IDDM), Addison's disease, celiac disease, chronic fatigue syndrome, colitis, Crohn's disease, fibromyalgia, hyperthyroidism, Graves disease, hypothyroidism, Hashimoto's disease, endometriosis, pernicious anemia, Goodpasture syndrome, Wegener's disease and rheumatic fever.

58. A method of treating an autoimmune disease in a subject in need thereof, the method comprising administering an effective amount of the tolerogenic dendritic cells of 55 to the subject.

59. A method of treating an autoimmune disease in a subject in need thereof, the method comprising administering an effective amount of tolerogenic dendritic cells to the subject, wherein the tolerogenic dendritic cells comprise physiological dendritic cells comprising material from an apoptotic dendritic cell obtained from the subject, an autoantigen, a fragment thereof, or a combination thereof.

60. The method of 58 or 59, wherein the autoimmune disease is selected from the group comprising multiple sclerosis, rheumatoid arthritis, juvenile rheumatoid arthritis, systemic lupus erythematosus, amyotrophic lateral sclerosis, pemphigus vulgaris, psoriasis, myasthenia gravis, thyroiditis, scleroderma, Sjogren's syndrome, thrombocytopenia purpura, cryoglobulinemia, autoimmune haemolytic anemia, insulin-dependent diabetes mellitus (IDDM), Addison's disease, celiac disease, chronic fatigue syndrome, colitis, Crohn's disease, fibromyalgia, hyperthyroidism, Graves disease, hypothyroidism, Hashimoto's disease, endometriosis, pernicious anemia, Goodpasture syndrome, Wegener's disease and rheumatic fever.

61. The method of 59 or 60, wherein the autoantigen is selected from the group comprising Rh blood group antigens, platelet integrin GpIIb:IIIa, noncollagenous domain of basement membrane collagen type IV, epidermal cadherin, streptococcal cell-wall antigens, rheumatoid factor IgG complexes with or without hepatitic C antigens, pancreatic β-cell antigen, myelin basic protein, proteolipid protein, myelin oligodendrocyte glycoprotein, desmoglein 3, glutamic acid decarboxylase, acetylcholine receptor, carboxypeptidase H, chromogranin A, glutamate decarboxylase, imogen-38, insulin, insulinoma antigen-2 and 2β, islet-specific glucose-6-phosphatase catalytic subunit related protein (IGRP), proinsulin, α-enolase, aquaporin-4, β-arrestin, S100-β, citrullinated protein, collagen II, heat shock proteins, human cartilage glycoprotein 39, La antigen, nucleosomal histones and ribonucleoproteins (snRNP), phospholipid-β-2 glycoprotein I complex, poly (ADP-ribose) polymerase, Sm antigens of U-1 small ribonucleoprotein complex, pancreatic islet cell antigens, cytoplasmic linker protein-170 (CLIP-170), Sjogren's syndrome antigen A (SS-A/Ro), Sjogren's syndrome antigen B (SS-B/La), Sjogren's lupus antigen (SL) and scleroderma antigen 70 (Scl-70)).

62. An ex vivo tolerogenic dendritic cell comprising material from an apoptotic dendritic cell obtained from a subject.

63. The ex vivo tolerogenic dendritic cell of 62, further comprising an autoantigen or a fragment thereof.

64. A composition comprising:
(a) a sample of dendritic cells obtained from a subject;
(b) an apoptotic agent; and
(c) an autoantigen or a fragment thereof.

65. The composition of 64, wherein the apoptotic agent comprises a psoralen, riboflavin-phosphate, or 5-aminolevulinic acid.

66. The composition of 65, wherein the psoralen is selected from the group comprising 8-MOP and amotosalen.

67. The composition of 66, wherein the psoralen is 8-MOP.

68. The ex vivo tolerogenic dendritic cell of 62 or 63, or the composition of any one of 64-67, wherein the autoantigen is selected from the group comprising Rh blood group antigens, platelet integrin GpIIb:IIIa, noncollagenous domain of basement membrane collagen type IV, epidermal cadherin, streptococcal cell-wall antigens, rheumatoid factor IgG complexes with or without hepatitic C antigens, pancreatic β-cell antigen, myelin basic protein, proteolipid protein, myelin oligodendrocyte glycoprotein, desmoglein 3, glutamic acid decarboxylase, acetylcholine receptor, carboxypeptidase H, chromogranin A, glutamate decarboxylase, imogen-38, insulin, insulinoma antigen-2 and 2β, islet-specific glucose-6-phosphatase catalytic subunit related protein (IGRP), proinsulin, α-enolase, aquaporin-4, β-arrestin, S100-β, citrullinated protein, collagen II, heat shock proteins, human cartilage glycoprotein 39, La antigen, nucleosomal histones and ribonucleoproteins (snRNP), phospholipid-β-2 glycoprotein I complex, poly (ADP-ribose) polymerase, Sm antigens of U-1 small ribonucleoprotein complex, pancreatic islet cell antigens, cytoplasmic linker protein-170 (CLIP-170), Sjogren's syndrome antigen A (SS-A/Ro), Sjogren's syndrome antigen B (SS-B/La), Sjogren's lupus antigen (SL) and scleroderma antigen 70 (Scl-70)).

1. A method to selectively produce tolerogenic dendritic cells, the method comprising the following steps:
a) providing dendritic cells from a donor,
b) exposing the dendritic cells of step a) to apoptotic agents;
c) providing physiologic dendritic cells from a recipient; and
d) combining the apoptotic donor dendritic cells of step b) with physiologic recipient dendritic cells from step c).

2. The method according to claim 1, wherein after step d), a step of co-incubating the apoptotic donor dendritic cells of step b) with the physiologic recipient dendritic cells of step c) is performed.

3. The method according to claim 2, wherein the step of co-incubating is performed for at least 0.5 hours (h), 1 h, 2 h, 3 h, 4 h, 5 h, or 6 h.

4. The method according to claim 1, wherein step d) takes place within the recipient.

5. The method according to claim 1, wherein the dendritic cells of step a) are derived from an extracorporeal blood sample of the donor.

6. The method according to claim 1, wherein the dendritic cells of step a) have been obtained by plate passage of peripheral blood mononuclear cells (PBMC) PBMC from the donor.

7. The method according to claim 1, wherein the apoptotic agents of step b) comprise a psoralen and UVA, riboflavin-phosphate and UVA, and/or 5-aminolevulinic acid and light, preferably wherein the psoralen is 8-MOP or amotosalen, and more preferably 8-MOP.

8.-9. (canceled)

10. The method according to claim 1, wherein the physiologic dendritic cells of step c) have been obtained by plate passage of PBMC from the recipient.

11. The method according to claim 1, wherein the donor and/or recipient are mammalian, preferably human.

12. A The method according to claim 1, wherein the donor of step a) is one genetic parent of a future recipient and the recipient of step c) is the other genetic parent of a future recipient.

13.-22. (canceled)

23. A method to selectively produce tolerogenic dendritic cells, the method comprising the following steps:
a) Providing dendritic cells from a recipient;
b) Exposing the dendritic cells of step a) to an apoptotic agent;
c) Providing physiologic dendritic cells from the recipient; and
d) Combining the apoptotic dendritic cells of step b) with the physiologic dendritic cells of step c).

24. The method according to claim 23, wherein after step d), a step of co-incubating the apoptotic dendritic cells of step b) with the physiologic dendritic cells of step c) is performed.

25. The method according to claim 24, wherein the step of co-incubating is performed for at least 0.5 h, 1 h, 2 h, 3 h, 4 h, 5 h, or 6 h.

26. The method according to claim 23, wherein step c) takes place within the recipient.

27. The method according to claim 23, wherein the dendritic cells of step a) are derived from an extracorporeal blood sample of the recipient.

28. The method according to claim 23, wherein the dendritic cells of step a) have been obtained by plate passage of PBMC from the recipient.

29. The method according to claim 23, wherein the apoptotic agents of step b) comprise a psoralen and UVA, riboflavin-phosphate and UVA, and/or 5-aminolevulinic acid and light, preferably wherein the psoralen is 8-MOP or amotosalen, and more preferably 8-MOP.

30.-31. (canceled)

32. The method according to claim 23, wherein the physiologic dendritic cells of step c) have been obtained by plate passage of PBMC from the recipient.

33. The method according to claim 23, wherein the donor and recipient are mammalian, preferably human.

34. Tolerogenic dendritic cells obtained by the method according to claim 1.

35. Tolerogenic dendritic cells obtained by the method according to claim 12.

36. Tolerogenic dendritic cells obtained by the method according to claim 23.

37. A method of preventing or reducing graft versus host disease in a subject in need thereof, comprising administering an effective amount of the tolerogenic dendritic cells according to claim 34.

38. A method to selectively produce tolerogenic dendritic cells, the method comprising the following steps:
a) Providing a first sample of dendritic cells obtained from a subject;
b) Exposing the dendritic cells of step a) to an apoptotic agent;
c) Providing a second sample of dendritic cells obtained from the subject; and
d) Combining the apoptotic dendritic cells of step b) with the dendritic cells of step c).

39. The method according to claim 38, wherein after step d), a step of co-incubating the apoptotic dendritic cells of step b) with the physiologic dendritic cells of step c) is performed.

40. The method according to claim 39, wherein the step of co-incubating is performed for at least 0.5 h, 1 h, 2 h, 3 h, 4 h, 5 h, or 6 h.

41. The method according to claim 38, wherein step c) of combining the apoptotic dendritic cells of step b) with the dendritic cells of step c) takes place within the subject.

42. The method according to claim 38, wherein the dendritic cells of step a) are derived from an extracorporeal blood sample of the subject.

43. The method according to claim 38, wherein the dendritic cells of step a) have been obtained by plate passage of PBMC from the subject.

44. The method according to claim 38, wherein the method further comprises step a1) of incubating the dendritic cells with an antigenic molecule.

45. The method according to claim 44, wherein the antigenic molecule is an autoantigen.

46. The method according to claim 44, wherein the antigenic molecule is derived from a natural source, chemically synthesized, or recombinantly produced.

47. The method according to claim 44, wherein the antigenic molecule is derived from a cell.

48. The method according to claim 45, wherein the autoantigen is a Rh blood group antigen, platelet integrin GpIIb:IIIa, noncollagenous domain of basement membrane collagen type IV, epidermal cadherin, streptococcal cell-wall antigens, rheumatoid factor IgG complexes with or without hepatitic C antigens, pancreatic β-cell antigen, myelin basic protein, proteolipid protein, myelin oligodendrocyte glycoprotein, desmoglein 3, glutamic acid decarboxylase, acetylcholine receptor, carboxypeptidase H, chromogranin A, glutamate decarboxylase, imogen-38, insulin, insulinoma antigen-2 and 2β, islet-specific glucose-6-phosphatase catalytic subunit related protein (IGRP), proinsulin, α-enolase, aquaporin-4, β-arrestin, S100-β, citrullinated protein, collagen II, heat shock proteins, human cartilage glycoprotein 39, La antigen, nucleosomal histones and ribonucleoproteins (snRNP), phospholipid-β-2 glycoprotein I complex, poly (ADP-ribose) polymerase, Sm antigens of U-1 small ribonucleoprotein complex, pancreatic islet cell antigens, cytoplasmic linker protein-170 (CLIP-170), Sjogren's syndrome antigen A (SS-A/Ro), Sjogren's syndrome antigen B (SS-B/La), Sjogren's lupus antigen (SL) or scleroderma antigen 70 (Scl-70)).

49. The method according to claim 38, wherein the apoptotic agents of step b) comprise a psoralen and UVA, riboflavin-phosphate and UVA, and/or 5-aminolevulinic acid and light, preferably wherein the psoralen is 8-MOP or amotosalen, and more preferably 8-MOP.

50.-51. (canceled)

52. The method according to claim 38, wherein the dendritic cells of step c) have been obtained by plate passage of PBMC from the subject.

53. The method according to claim 38, wherein the subject is mammalian, preferably human.

54. Tolerogenic dendritic cells obtained by the method according to claim 38.

55. A method of treating an autoimmune disease in a subject in need thereof, comprising administering to the subject an effective amount of the tolerogenic dendritic cells according to claim 54.

56. The method according to claim 55, wherein the autoimmune disease is multiple sclerosis, rheumatoid arthritis, juvenile rheumatoid arthritis, systemic lupus erythematosus, amyotrophic lateral sclerosis, pemphigus vulgaris, psoriasis, myasthenia gravis, thyroiditis, scleroderma, Sjogren's syndrome, thrombocytopeniapurpura, cryoglobulinemia, autoimmune haemolytic anemia, insulin-dependent diabetes mellitus (IDDM), Addison's disease, celiac disease, chronic fatigue syndrome, colitis, Crohn's disease, fibromyalgia, hyperthyroidism, Graves disease, hypothyroidism, Hashimoto's disease, endometriosis, pernicious anemia, Goodpasture syndrome, Wegener's disease, or rheumatic fever.

57. (canceled)

58. The method of claim 55, comprising administering an effective amount of tolerogenic dendritic cells to the subject, wherein the tolerogenic dendritic cells comprise physiological dendritic cells comprising material from an apoptotic dendritic cell obtained from the subject, an autoantigen, a fragment thereof, or a combination thereof.

59. The method of claim 58, wherein the autoimmune disease is multiple sclerosis, rheumatoid arthritis, juvenile rheumatoid arthritis, systemic lupus erythematosus, amyotrophic lateral sclerosis, pemphigus vulgaris, psoriasis, myasthenia gravis, thyroiditis, scleroderma, Sjogren's syndrome, thrombocytopenia purpura, cryoglobulinemia, autoimmune haemolytic anemia, insulin-dependent diabetes mellitus (IDDM), Addison's disease, celiac disease, chronic fatigue syndrome, colitis, Crohn's disease, fibromyalgia, hyperthyroidism, Graves disease, hypothyroidism, Hashimoto's disease, endometriosis, pernicious anemia, Goodpasture syndrome, Wegener's disease, or rheumatic fever.

60. The method of claim 58, wherein the autoantigen is an Rh blood group antigen, platelet integrin GpIIb:IIIa, noncollagenous domain of basement membrane collagen type IV, epidermal cadherin, streptococcal cell-wall antigens, rheumatoid factor IgG complexes with or without hepatitic C antigens, pancreatic β-cell antigen, myelin basic protein, proteolipid protein, myelin oligodendrocyte glycoprotein, desmoglein 3, glutamic acid decarboxylase, acetylcholine receptor, carboxypeptidase H, chromogranin A, glutamate decarboxylase, imogen-38, insulin, insulinoma antigen-2 and 2β, islet-specific glucose-6-phosphatase catalytic subunit related protein (IGRP), proinsulin, α-enolase, aquaporin-4, β-arrestin, S100-β, citrullinated protein, collagen II, heat shock proteins, human cartilage glycoprotein 39, La antigen, nucleosomal histones and ribonucleoproteins (snRNP), phospholipid-β-2 glycoprotein I complex, poly (ADP-ribose) polymerase, Sm antigens of U-1 small ribonucleoprotein complex, pancreatic islet cell antigens, cytoplasmic linker protein-170 (CLIP-170), Sjogren's syndrome antigen A (SS-A/Ro), Sjogren's syndrome antigen B (SS-B/La), Sjogren's lupus antigen (SL), or scleroderma antigen 70 (Scl-70)).

61. An ex vivo tolerogenic dendritic cell comprising material from an apoptotic dendritic cell obtained from a subject.

62. The ex vivo tolerogenic dendritic cell of claim 61, further comprising an autoantigen or a fragment thereof.

63. A composition comprising:
(a) a sample of dendritic cells obtained from a subject;
(b) an apoptotic agent; and
(c) an autoantigen or a fragment thereof.

64. The composition of claim 63, wherein the apoptotic agent is a psoralen, riboflavin-phosphate, or 5-aminolevulinic acid, preferably wherein the psoralen is 8-MOP or amotosalen, and more preferably 8-MOP.

65.-66. (canceled)

67. The ex vivo tolerogenic dendritic cell of claim 61, wherein the autoantigen is a Rh blood group antigen, platelet integrin GpIIb:IIIa, noncollagenous domain of basement membrane collagen type IV, epidermal cadherin, streptococcal cell-wall antigens, rheumatoid factor IgG complexes with or without hepatitic C antigens, pancreatic β-cell antigen, myelin basic protein, proteolipid protein, myelin oligodendrocyte glycoprotein, desmoglein 3, glutamic acid decarboxylase, acetylcholine receptor, carboxypeptidase H, chromogranin A, glutamate decarboxylase, imogen-38, insulin, insulinoma antigen-2 and 2β, islet-specific glucose-6-phosphatase catalytic subunit related protein (IGRP), proinsulin, α-enolase, aquaporin-4, β-arrestin, S100-β, citrullinated protein, collagen II, heat shock proteins, human cartilage glycoprotein 39, La antigen, nucleosomal histones and ribonucleoproteins (snRNP), phospholipid-β-2 glycoprotein I complex, poly (ADP-ribose) polymerase, Sm antigens of U-1 small ribonucleoprotein complex, pancreatic islet cell antigens, cytoplasmic linker protein-170 (CLIP-170), Sjogren's syndrome antigen A (SS-A/Ro), Sjogren's syndrome antigen B (SS-B/La), Sjogren's lupus antigen (SL), or scleroderma antigen 70 (Scl-70)).

68. A method of preventing or reducing graft versus host disease in a subject in need thereof, comprising administering an effective amount of the tolerogenic dendritic cells according to claim 36.
